SEQUENCE LISTING (19 sequences)

SEQ ID NO: 1 - DNA, 89 bp, Artificial Sequence
  Synthetic construct
  misc_feature (44)..(57): n is a, c, g, or t
gaaacggagacggataagacacaaaaaaacaaacaaaaaccccnnnnnnnnnnnnnngtaaagggaaaacaatgatacgaacacgcata

SEQ ID NO: 2 - DNA, 20 bp, Artificial Sequence
  Synthetic construct
gaaacggagacggataagac

SEQ ID NO: 3 - DNA, 20 bp, Artificial Sequence
  Synthetic construct
tatgcgtgttcgtatcattg

SEQ ID NO: 4 - DNA, 89 bp, Artificial Sequence
  Synthetic construct
  misc_feature (33)..(46): n is a, c, g, or t
tattgcgatagctgagagagaagacgcgagggnnnnnnnnnnnnnngcgaaaacaaaaaacaaaaataagaatccaagcagcagcaaca

SEQ ID NO: 5 - DNA, 20 bp, Artificial Sequence
  Synthetic construct
tattgcgatagctgagagag

SEQ ID NO: 6 - DNA, 19 bp, Artificial Sequence
  Synthetic construct
gttgctgctgcttggattc

SEQ ID NO: 7 - DNA, 41 bp, Artificial Sequence
  Synthetic construct
aagacctacctcacatggccaacactcggacaaaaaaaaaa

SEQ ID NO: 8 - DNA, 10 bp, Artificial Sequence
  Synthetic construct
tttttttttt

SEQ ID NO: 9 - DNA, 45 bp, Artificial Sequence
  Synthetic construct
tccctacacgacgctcttccgatcttattgcgatagctgagagag

SEQ ID NO: 10 - DNA, 44 bp, Artificial Sequence
  Synthetic construct
gttcagacgtgtgctcttccgatctgttgctgctgcttggattc

SEQ ID NO: 11 - DNA, 45 bp, Artificial Sequence
  Synthetic construct
tccctacacgacgctcttccgatctgaaacggagacggataagac

SEQ ID NO: 12 - DNA, 45 bp, Artificial Sequence
  Synthetic construct
gttcagacgtgtgctcttccgatcttatgcgtgttcgtatcattg

SEQ ID NO: 13 - DNA, 68 bp, Artificial Sequence
  Synthetic construct
aatgatacggcgaccaccgagatctacacatcacgacactctttccctacacgacgctcttccgatct

SEQ ID NO: 14 - DNA, 64 bp, Artificial Sequence
  Synthetic construct
caagcagaagacggcatacgagatacagtggtgactggagttcagacgtgtgctcttccgatct

SEQ ID NO: 15 - DNA, 64 bp, Artificial Sequence
  Synthetic construct
caagcagaagacggcatacgagatgccaatgtgactggagttcagacgtgtgctcttccgatct

SEQ ID NO: 16 - DNA, 64 bp, Artificial Sequence
  Synthetic construct
caagcagaagacggcatacgagatcagatcgtgactggagttcagacgtgtgctcttccgatct

SEQ ID NO: 17 - DNA, 64 bp, Artificial Sequence
  Synthetic construct
caagcagaagacggcatacgagatacttgagtgactggagttcagacgtgtgctcttccgatct

SEQ ID NO: 18 - DNA, 16 bp, Artificial Sequence
  Synthetic construct
tccgtttctgttgctg

SEQ ID NO: 19 - DNA, 10 bp, Artificial Sequence
  Synthetic construct
caacagaaac

(19) United States
(12) Patent Application Publication
(10) Pub. No.: US 2024/0175009 A1
(43) Pub. Date: May 30, 2024

(54) NEXTGEN CHEMOMETRICS USING PROXIMITY LIGATION ASSAY (PLA)

(71) Applicant: BOARD OF REGENTS, THE UNIVERSITY OF TEXAS SYSTEM, Austin, TX (US)

(72) Inventors: Andrew ELLINGTON, Austin, TX (US); Sanchita BHADRA, Austin, TX (US); Zachary WENTZELL, Austin, TX (US)

(21) Appl. No.: 18/285,329

(22) PCT Filed: Apr. 4, 2022
(86) PCT No.: PCT/US2022/023361

§ 371 Date: Oct. 2, 2023

Related U.S. Application Data
(60) Provisional application No. 63/170,063, filed on Apr. 2, 2021.

(51) Int. Cl. C12N 15/10; G16B 40/00

(52) U.S. Cl. CPC C12N 15/1068; G16B 40/00

(57) ABSTRACT

The present invention relates to compositions and methods for material characterization using probe libraries. The methods include steps of unbiased sensing with probe libraries (e.g., oligonucleotide probes, or conjugated probes that comprise peptides conjugated to polynucleotide barcodes) that are followed with identifying informative patterns of the bounded probes when the probed libraries are applied to a material. In some examples, the probes used herein comprise binding moieties composed of random sequences (e.g., random peptide sequences or random polynucleotide sequences) that enable the unbiased sensing of the substrates in a sample for the determination of known and unknown materials. In some examples, the methods herein further comprise analyzing the sequences of the bounded oligonucleotide probes or the bounded conjugated probes and determining the patterns of the sequences that is indicative of the substrates bounded by the probes.

### I. CROSS-REFERENCE TO RELATED APPLICATIONS

This application claims the benefit of U.S. Provisional Application No. 63/170,063, filed Apr. 2, 2021, which is expressly incorporated herein by reference in its entirety.

### II. STATEMENT REGARDING FEDERALLY SPONSORED RESEARCH OR DEVELOPMENT

This invention was made with government support under Grant no. DMR1720595 awarded by the National Science Foundation and Grant no. 80NSSC18K1140 awarded by the National Aeronautics and Space Administration (NASA). The government has certain rights in the invention.

### III. BACKGROUND

Most strategies for detection reply upon reagents specific to a known target. However, such regents may not be present when exploring an unknown substrate on Earth or other planets or studying new disease markers. What are needed are new compositions and methods for material characterization.

### IV. SUMMARY

The methods disclosed herein relate to a PLA-based assay for materials characterization. The methods include steps of unbiased sensing with probe libraries (e.g., oligonucleotide probes, or conjugated probes that comprise peptides conjugated to polynucleotide barcodes) that are followed with identifying informative patterns of the bounded probes when the probed libraries are applied to a material. In some examples, the probes used herein comprise binding moieties composed of random sequences (e.g., random peptide sequences or random polynucleotide sequences) that enable the unbiased sensing of the substrates in a sample for the determination of known and unknown materials. In some examples, the methods herein further comprise analyzing the sequences of the bounded oligonucleotide probes or the bounded conjugated probes and determining the patterns of the sequences that is indicative of the substrates bounded by the probes.

Accordingly, in some aspects, disclosed herein is a method of detecting a target in a sample, said method comprising:
obtaining a first library of probes and/or a second library of probes, wherein each probe of the first library comprises a 5′ primer binding site, a binding moiety, and a 3′ splinting site, wherein each probe of the second library comprises a 5′ splinting site, a binding moiety, and a 3′ primer binding site;
mixing the probes of the first library and the probes of the second library with the sample thereby allowing binding of the probes through their binding moieties to one or more substrates on the target;
ligating the 3′ splinting site of the probe of the first library and the splinting site of the probe of the second library thereby producing a ligated probe template;
performing amplification of the ligated probe template thereby producing a plurality of amplicons; and
sequencing the plurality of amplicons.

In some embodiments, the method further comprises washing the sample before the step of ligation thereby removing the unbound probes.

In some embodiments, the binding moiety is a peptide, a nucleic acid, or an oligourethane. In some embodiments, the binding moiety comprises a random sequence.

In some embodiments, the sequence of the primer binding site is unique to the probe. In some embodiments, the probe further comprises a unique polynucleotide barcode.

In some embodiments, the binding moiety specifically binds to an organic or inorganic substrate.

In some embodiments, the splinting site is about 8 nucleotides in length. In some embodiments, the splinting site is single stranded, a duplex, or a hemiduplex.

In some embodiments, the method of any preceding aspect further comprises adding an oligonucleotide splint to the sample, wherein said oligonucleotide splint comprises a first region and a second region, wherein the first region is complementary to the 3′ splinting site of the probe of the first library and the second region is complementary to the 5′ splinting site of the probe of the second library.

In some embodiments, the method of any preceding aspect further comprises identifying and quantifying the sequenced data. In some embodiments, the step of identifying and quantifying the sequenced data comprises extracting the sequences (e.g., the barcode sequences and/or the binding moiety sequences) from the sequenced amplicons.

In some embodiments, the method further comprises
determining a plurality of k-mers of each sequence;
determining the counts of each k-mer; and
calculating the p-value by comparing the frequency of each k-mer in the sample with a reference control.

In some embodiments, the method further comprises translating the quantitative results into a graphic pattern. In some embodiments, the method further comprises comparing the graphical pattern to a reference control thereby identifying the target.

In some embodiments, the method further comprises processing the qualified sequencing data using a dimensionality reduction algorithm.

Also disclosed herein is a method of detecting a biotic substrate in a sample, said method comprising:
obtaining a first library of probes and/or a second library of probes, wherein each probe of the first library comprises a 5′ primer binding site, a binding moiety, and a 3′ splinting site, and wherein each probe of the second library comprises a 5′ splinting site, a binding moiety, and a 3′ primer binding site;
mixing the probes of the first library and the probes of the second library with the sample thereby allowing binding of the oligonucleotide probes through their binding moieties to one or more substrates in the sample;
ligating the 3′ splinting site of the probe of the first library and the 5′ splinting site of the probe of the second library thereby producing a ligated probe template;
performing amplification of the ligated probe template thereby producing a plurality of amplicons;
sequencing the plurality of amplicons;
identifying and quantifying the sequenced data;
comprising translating the quantitative results into a graphic pattern; and
determining that the sample has biotic substrate if the graphical pattern of the sample comprises a pattern of a biotic substrate.

Also disclosed herein is a method of diagnosing a cancer in a subject, said method comprising:
obtaining a biological sample from the subject;
obtaining a first library of probes and/or a second library of probes, wherein each probe of the first library comprises a 5′ primer binding site, a binding moiety, and a 3′ splinting site, each probe of the second library comprises a 5′ splinting site, a binding moiety, and a 3′ primer binding site;
mixing the first library of probes and the second library of probes with the biological sample thereby allowing binding of the probes through their binding moieties to one or more substrates in the biological sample;
ligating the 3′ splinting site of the probe of the first library and the 5′ splinting site of the probe of the second library thereby producing a ligated probe template;
performing amplification of the ligated probe template thereby producing a plurality of amplicons;
sequencing the plurality of amplicons;
identifying and quantifying the sequenced data;
translating the quantitative results into a graphic pattern; and
determining that the subject has cancer if the graphical pattern comprises a pattern of a cancer biomarker.

A method of determining spatial position of one or more substrate on a surface, said method comprising:
obtaining a first library of probes and/or a second library of probes, wherein each probe of the first library comprises a 5′ primer binding site, a binding moiety, and a 3′ splinting site, and wherein each probe of the second library comprises a 5′ splinting site, a binding moiety, and a 3′ primer binding site;
applying the first library of probes and the second library of probes to the surface thereby allowing binding of the probes through their binding moieties to one or more substrates on the surface;
ligating the 3′ splinting site of the probe of the first library and the 5′ splinting site of the probe of the second library thereby producing a ligated probe template;
performing amplification of the ligated probe template thereby producing a plurality of amplicons;
sequencing the plurality of amplicons;
identifying and quantifying the sequenced data; and
determining the spatial position of the one or more substrate.

### V. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows schematic depicting Proximity ligation assay for material analysis.

FIG. 2 shows schematic depicting the use of a single stranded DNA as proximity substrate.

FIG. 3 shows a flowchart depicting the N15 extraction pipeline used to process NGS data.

FIG. 4A shows converting a k-mer (even k) to a pair of coordinates for plotting.

FIG. 4B shows sample k-mer fingerprint with each k-mer colored by the significance of its enrichment. P-values are show as log(p-value).

FIGS. 5A and 5B show 2D histograms of median log 2 k-mer counts versus the coefficient of log 2 k-mer counts grouped by analyte. A drastic effect is observed on the distribution of median counts between (FIG. 5A) for 6-mers and (FIG. 5B) for 8-mers. As the length of k-mers is increased, counts become more variable and decrease for most k-mers. FIG. 5C shows a similar effect by considering the distribution of effective substrate specificity (ESS) for highly enriched, low-variance k-mers. For 5-mers, almost all k-mers are highly enriched and low-variance (similar to (FIG. 5A) for 6-mers), but they're also non-specific with ESS mostly around 5 (the number of different analytes). However, as the length of the k-mers is increased, new peaks start to emerge as the ESS distribution starts to shift toward higher specificity. By k-mers of length 10, nearly all highly enriched, low-variancek-mers are specific to a single type of analyte. This effect is important to consider when choosing a value of k for future analysis. NKC—normalized k-mer count: Mag—magnetic beads.

In FIG. 6A, because of the noise present in the data and the small number of samples, PCA can choose non-informative features when mapping to a new set of basis vectors. Here the study used 8-mers. Thus, the analysis started with 65,536 features and were trying to reduce them down to 2 meaningful features using only 21 data points. It has difficulty partitioning the different analytes. In FIG. 6B, to remove most of the noisy k-mers, the analysis filtered down to a set of 267 k-mers with enrichment p-values (between the analyte replicates and all other samples: Mann-Whitney U test)<0.02 for multiple analytes. This is similar to choosing a set of lower entropy k-mers, but requiring the k-mer to interact with multiple analytes (ESS>=2). Using the filtered set of k-mers, our model more clearly groups together samples by analyte. Moreover, the samples of the two proteins are also close to each other while the plastic, silica, and chitin samples are farther away. This gives evidence that the principal components are meaningful and capture useful relationships from the data.

FIG. 7: (Left) Further illustrating the partitioning achieved by the PCA model in (Right) by fitting a simple kNN classifier to the PCA data. (Right) Improved partitioning of each of the analytes achieved by using 10-mers. With well-defined separation between analytes, new points are mapped to the PCA space and inferences are make about which analyte they may contain.

FIGS. 8A-8B show schematic depicting peptide PLA methodology. FIG. 8A shows setup of positive control peptide PLA using Strep Tag II peptide and magnetic streptavidin beads. FIG. 8B shows barcoded peptide PLA library and its readout using barcode-specific primer pairs.

FIG. 9 shows unbiased ‘sensing’ of material complexity, material classification and identification of potential to exhibit properties associated with life.

FIG. 10 shows probe design for proximity dependent ligation assays.

FIG. 11 shows solution phase and particulate surface bound PLA optimization schematic

FIG. 12 shows PLA protocols optimized to maximize specific signal.

FIG. 13 shows PLA analysis of particulate materials using DNA libraries—cloning and Sanger sequencing

FIG. 14 shows NextGen sequence analysis of PLA samples.

FIG. 15 shows statistical analysis and data visualization.

FIG. 16 shows distinct patterns emerge in the DNA sequence space enriched by different materials.

FIGS. 17A and 17B show signature DNA sequences found to uniquely associate with certain materials.

FIG. 18 shows non-parametric analyses also partition PLA sequence data into material-specific clusters.

FIG. 19 shows development of experimental methods and analytical pipelines.

FIG. 20 shows data visualization. For even numbered k-mers (4-mers, 6-mers, 8-mers, etc.), the k-mer is split into a left and right half. Bases are encoded into numbers (G=1, A=2, T=3, C=4) and sorted in ascending order. A ranking is then applied to the ordering to create a contiguous mapping from k-mer halves to numbers. These mappings can be used as axes for heatmaps and other visualizations.

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10)” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims that follow, reference will be made to a number of terms which shall be defined to have the following meanings:

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used herein, the term “aptamer” refers to an oligonucleotide that has been designed or discovered that is able to specifically bind a target sequence.

“Optional” or “optionally” means that the subsequently described event or circumstance may or may not occur, and that the description includes instances where said event or circumstance occurs and instances where it does not.

The term “biocompatible” generally refers to a material and any metabolites or degradation products thereof that are generally non-toxic to the recipient and do not cause significant adverse effects to the subject.

“Biological sample” refers to a sample of biological material obtained from a subject. Biological samples include all clinical samples useful for detection of disease or disorder in subjects. Appropriate samples include any conventional biological samples, including clinical samples obtained from a human or veterinary subject. Exemplary samples include, without limitation, cells, cell lysates, blood smears, cytocentrifuge preparations, cytology smears, bodily fluids, tissue biopsies or autopsies, fine-needle aspirates, and/or tissue sections.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, reagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, reagents, conjugates, active metabolites, isomers, fragments, analogs, etc.

A “control” is an alternative subject or sample used in an experiment for comparison purposes.

“Diagnosis” refers to the process of identifying a disease by its signs, symptoms and results of various tests. The conclusion reached through that process is also called “a diagnosis.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

Nucleic acid: A deoxyribonucleotide or ribonucleotide polymer, which can include analogues of natural nucleotides that hybridize to nucleic acid molecules in a manner similar to naturally occurring nucleotides. In a particular example, a nucleic acid molecule is a single stranded (ss) DNA or RNA molecule, such as a probe or primer. In another particular example, a nucleic acid molecule is a double stranded (ds) nucleic acid, such as a target nucleic acid. Examples of modified nucleic acids are those with altered sugar moieties, such as a locked nucleic acid (LNA).

Nucleotide: The fundamental unit of nucleic acid molecules. A nucleotide includes a nitrogen-containing base attached to a pentose monosaccharide with one, two, or three phosphate groups attached by ester linkages to the saccharide moiety. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

The term “polynucleotide” refers to a single or double stranded polymer composed of nucleotide monomers (DNA or RNA).

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

Sequence identity: The similarity between two nucleic acid sequences is expressed in terms of the similarity between the sequences, otherwise referred to as sequence identity. Sequence identity is frequently measured in terms of percentage identity, similarity, or homology: a higher percentage identity indicates a higher degree of sequence similarity. The NCBI Basic Local Alignment Search Tool (BLAST), Altschul et al, J. Mol. Biol. 215:403-10, 1990, is available from several sources, including the National Center for Biotechnology Information (NCBI, Bethesda, MD), for use in connection with the sequence analysis programs blastp, blastn, blastx, tblastn and tblastx. It can be accessed through the NCBI website. A description of how to determine sequence identity using this program is also available on the website. When less than the entire sequence is being compared for sequence identity, homologs will typically possess at least 75% sequence identity over short windows of 10-20 amino acids, and can possess sequence identities of at least 85% or at least 90% or 95% depending on their similarity to the reference sequence. Methods for determining sequence identity over such short windows are described on the NCBI website. These sequence identity ranges are provided for guidance only: it is entirely possible that strongly significant homologs could be obtained that fall outside of the ranges provided.

Subject: Any mammal, such as humans, non-human primates, pigs, sheep, horses, dogs, cats, cows, rodents and the like. In two non-limiting examples, a subject is a human subject or a murine subject.

The terms “treat,” “treating.” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset, during early onset, or after an established development of a disorder or symptoms thereof. Prophylactic administration can occur for several days to years prior to the manifestation of symptoms of a disorder.

“Therapeutically effective amount” or “therapeutically effective dose” of a composition (e.g. a composition comprising an agent) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In the present invention, “specific for” and “specificity” mean selective binding. A probe may be “target-specific” in that it binds or interacts with its targets above detectable noise in a sample. The term “binding.” and “specific binding” are used interchangeably to refer to the ability of a reagent to selectively bind its target. Typically, specificity is characterized by a dissociation constant of 10⁴ M⁻¹ to 10¹² M⁻¹. Empirical methods using appropriate controls may be employed to distinguish specific and non-specific binding in a particular case.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods

The methods include steps of unbiased sensing with probe libraries (e.g., ligated or unligated probes, nucleic acid probes or peptide probes conjugated to nucleic acid barcodes) that are followed with identifying informative patterns of the bounded probes binding proportions when the probed libraries are applied to a material.

In some aspects, provided herein is an agnostic method to characterize materials based on the proximity ligation assay with probe libraries. By transducing binding information into ligation, and hence into amplicons, the methods generate next generation sequencing (NGS) data for materials characterization. Such NGS data is a generalizable fingerprint, and the use of NGS on spaceflights or in extraterrestrial probes can be readily contemplated.

Accordingly, in some aspects, disclosed herein is a method of detecting a target in a sample, said method comprising:
obtaining a first library of probes and/or a second library of probes, wherein each probe of the first library comprises a 5′ primer binding site, a binding moiety, and a 3′ splinting site, wherein each probe of the second library comprises a 5′ splinting site, a binding moiety, and a 3′ primer binding site; and
mixing the probes of the first library and/or the probes of the second library with the sample thereby allowing binding of the probes through their binding moieties to one or more substrates on the target:

In some embodiments, the method further comprises
ligating the 3′ splinting site of the probe of the first library and the splinting site of the probe of the second library thereby producing a ligated probe template;
performing amplification of the ligated probe template thereby producing a plurality of amplicons; and
sequencing the plurality of amplicons.

Proximity ligation assay (PLA) is a variant of immunoPCR in which only adjacent binding events are amplified. In the assay, two affinity probes bearing oligonucleotide tails bound to adjacent sites on an organic or inorganic surface are ligated together to form a unique amplicon detectable by an amplification assay (e.g., PCR). Because the assay relies on two specific binding events, the level of background (target independent) ligation is low. Further, the requirement for two oligonucleotides to be brought together by a bridge reduces background dramatically and is a primary driver for increased sensitivity.

In some embodiments, the binding moiety of the probe disclosed herein is a non-nucleic acid probe (e.g., peptide or oligourethane). In some embodiments, the probe comprises a peptide or oligourethane. In some embodiments, the binding moiety is a peptide about at least 4, 6, 8, 10, 12, 14, 16, 18, 20, 25, 30, 35, 40, 45, 50, 55, 60, 65, 70, 75, 80, 85, 90, 95, or 100 amino acids in length. In some embodiments, the binding moiety is a peptide about 50 to 200 amino acids in length. In some embodiments, the binding moiety is a peptide about 50 to 500 amino acids in length. In some embodiments, the binding moiety is a peptide about 100 to 1000 amino acids in length. In some embodiments, the probe further comprises a polynucleotide barcode, wherein the polynucleotide barcode is linked to the peptide or the oligourethane (e.g., in FIG. 8 shown herein). In some embodiments, the polynucleotide barcode linked to the peptide is about 4, 6, 8, 10, 12, 14, 16, 18, 20, 25, 30, 35, 40, 45, 50, 55, 60, 65, 70, 75, 80, 85, 90, 95, 100, 120, 140, 160, 180, 200, 250, 300, 350, 400, or 500 nucleotides in length. In some embodiments, the polynucleotide barcode linked to the peptide is about 5 to 100 nucleotides in length. In some embodiments, the polynucleotide barcode linked to the peptide is about 50 nucleotides in length. It should be understood and herein contemplated that the polynucleotide barcode linked to the peptide is unique to the probe.

In some embodiments, the binding moiety of the probe disclosed herein is a nucleic acid (e.g., DNA or RNA). In some embodiments, the binding moiety is about 5, 10, 15, 20, 25, 30, 35, 40, 45, 50, 55, 60, 65, 70, 75, 80, 85, 90, 95, 100, 120, 140, 160, 180, or 200 nucleotides in length. In some embodiments, the binding moiety is about 10-50 nucleotides in length. In some embodiments, the binding moiety is about 15 nucleotides in length. In some embodiments, the binding moiety is an aptamer.

As used herein, the term “aptamer” refers to nucleic acids having a desirable action on a target (e.g., binding of the target). In some examples, aptamers have a variety of shapes due to their tendency to form helices and single-stranded loops. They can bind targets with high selectivity and specificity. Aptamer binding can be determined by its tertiary structure. Target recognition and binding involve three-dimensional, shape-dependent interactions as well as hydrophobic interactions, base-stacking, and intercalation.

The term “library of probes” herein refers to probes that comprise randomly generated sequences. In some embodiments, the binding moiety comprise a random sequence (e.g., a random peptide sequence or a random nucleic acid sequence). In some embodiments, the binding moiety comprises a random peptide sequence (e.g., about 4, 6, 8, 10, 12, 14, 16, 18, 20, 25, 30, 35, 40, 45, 50, 55, 60, 65, 70, 75, 80, 85, 90, 95, or 100 amino acids in length). In some embodiments, the binding moiety comprises a random nucleic acid sequence (e.g., about 5, 10, 15, 20, 25, 30, 35, 40, 45, 50, 55, 60, 65, 70, 75, 80, 85, 90, 95, 100, 120, 140, 160, 180, or 200 nucleotides in length). Accordingly, the method disclosed herein include the uses of unbiased sensing with libraries of probes without known targets.

The targets detected by the binding moiety described herein can be a protein, protein complex, peptide, antibody, carbohydrate, nucleic acid, cell or a receptor. In some embodiments, the target specifically bounded by the binding moiety is an organic substrate or an inorganic substrate. In some embodiments, the method disclosed herein is performed on a molecular surface or in solution. In some embodiments, the molecular surface is organic or inorganic. In some embodiments, the molecular surface comprises crystals, plastic, or metal. In some embodiments, the cell is a mammalian cell, cancer cell, or cell infected with an infectious agent.

The term “sample” herein refers to a material obtained or isolated from a fresh or preserved biological sample or synthetically-created source that contains molecules of interest. Samples can include at least one cell, fetal cell, cell culture, tissue specimen, blood, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. Samples can also include non-human sources, such as non-human primates, rodents and other mammals.

In some embodiments, the method disclosed herein further comprises washing the sample before the step of ligation thereby removing the unbound probes. In some embodiments, the method does not comprise a washing step and is performed from mixing the probes with the samples to ligation.

In some embodiments, the oligonucleotide probe further comprises an extension site. In some embodiments, the extension site is single-stranded polynucleotide. In some embodiments, the extension is located between the primer binding site and the splinting site and adjusts the probe length by adjusting the length of the primer binding site length or random region length.

In some embodiments, the splinting site is about 3, 5, 7, 9, 11, 13, 15, 17, 19, 21, 23, 25, 27, 29, 31, 33, 35, 37, 39, 41, 43, 45, 47, or 50 nucleotides in length. In some embodiments, the splinting site is about 3-15 nucleotides in length. In some embodiments, the splinting site is about 8 nucleotides in length. The splinting site can be single stranded, a duplex, or a hemiduplex. The splinting site can be blocked by forming a hemiduplex or a duplex that then needs to be strand displaced by a splint oligonucleotide.

In some embodiments, the method herein can also include the step of adding an oligonucleotide splint to the sample, wherein said oligonucleotide splint comprises a first region and a second region, wherein the first region is complementary to the 3′ splinting site of the probe of the first library and the second region is complementary to the 5′ splinting site of the probe of the second library. The oligonucleotide splint thereby ligates the probes thereby producing a ligated probe template. In some embodiments, the oligonucleotide splint is about 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 nucleotides in length. In some embodiments, the oligonucleotide splint is about 16 nucleotides in length. In some embodiments, the ligation is with a T4 DNA ligase. In some embodiments, for ligated nucleic acid probe, the ligated probe template is amplified with polymerase chain reaction amplification. In some embodiments, for the non-nucleic acid probe, the polynucleotide barcodes and/or the primer binding sites of the ligated probe template is amplified with polymerase chain reaction amplification.

“Polymerase chain reaction,” or “PCR,” generally refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following processes: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each process in a thermal cycler instrument. Particular temperatures, durations at each process, and rates of change between processes depend on many factors, e.g., exemplified by the references: McPherson et al., editors, PCR: A Practical Approach and PCR2: A Practical Approach (IRL Press, Oxford, 1991 and 1995, respectively).

The present invention includes a method for detecting a target in a sample, said method comprising mixing probes of a first library and probes of a second library with the sample thereby allowing binding of the probe through its binding moiety to a substrate on the target; adding at least one oligonucleotide splint to the sample, wherein said oligonucleotide splint comprises a first region and a second region, wherein the first region is complementary to the 3′ splinting site of the probe of the first library and the second region is complementary to the 5′ splinting site of the probe of the second library: ligating the probes to the nucleic acid splint thereby producing a ligated probe template; performing amplification to produce a plurality of ligated amplicons; and sequencing the plurality of ligated oligonucleotide amplicons. In one aspect, the ligation is via a protein ligase, such as T4 DNA ligase, chemical ligation or a nucleic acid ligase such as a ribozyme or deoxyribozyme. The ligation can be accomplished with a template independent ligase, e.g., a T4 RNA ligase 1 or 2.

Any technique for sequencing nucleic acids can be used in the methods of the present disclosure. DNA sequencing techniques include dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during polymerization, and SOLID sequencing. The input RNA may be 10%, 15%, 30%, or higher.

In certain embodiments, the sequencing technique used in the methods of the provided disclosure generates at least 100 reads per run, at least 200 reads per run, at least 300 reads per run, at least 400 reads per run, at least 500 reads per run, at least 600 reads per run, at least 700 reads per run, at least 800 reads per run, at least 900 reads per run, at least 1000 reads per run, at least 5,000 reads per run, at least 10,000 reads per run, at least 50,000 reads per run, at least 100,000 reads per run, at least 500,000 reads per run, at least 1,000,000 reads per run, at least 2,000,000 reads per run, at least 3,000,000 reads per run, at least 4,000,000 reads per run at least 5000,000 reads per runs at least 6,000,000 reads per run at least 7,000,000 reads per run at least 8,000,000 reads per runs at least 9,000,000 reads per run, at least 10,000,000 reads per run, or more. In certain embodiments, the sequencing technique used in the methods of the provided disclosure can generate at least about 30 bp, about 40 bp, about 50 bp, about 60 bp, about 70 bp, about 80 bp, about 90 bp, about 100 bp, about 110, about 120 by per read, about 150 bp, about 200 bp, about 250 bp, about 300 bp, about 350) bp, about 400 bp, about 450 bp, about 500 bp, about 550 bp, about 600 bp, about 700 bp, about 800 bp, about 900 bp, about 1,000 bp, or more per read. For example, the sequencing technique used in the methods of the provided disclosure can generate at least about 30 bp. 40 bp, 50 bp. 60 bp, 70 bp, 80 bp, 90 bp. 100 bp. 110 bp, 120 bp, 150 bp. 200 bp, 250 bp. 300 bp, 350 bp. 400 bp, 450 bp. 500 bp, 550 bp. 600 bp, 650 bp, 700 bp. 750 bp. 800 bp, 850 bp. 900 bp, 950 bp, 1,000 bp, or more by per read.

In some embodiments, the sequencing technologies used in the methods of the present disclosure HiSeg™ and MiSeg™, True Single Molecule Sequencing. 454 Sequencing, Genome Sequencer FLX™, SOLID™ Sequencing, Ion Torrent™ Sequencing, SMRT™ Sequencing. Nanopore Sequencing. Methods for sequencing are known in the art. See, e.g., U.S. Patent Publication No: 20210132076, incorporated by reference herein in its entirety.

In some examples, the probes used herein are not composed of known aptamers with known binding preferences. In some examples, binding can also be achieved using libraries of non-nucleic acid binding moieties, such as peptides, that do not necessarily contain peptides directed at specific known targets. Accordingly, to determining the detection of a target, the method disclosed herein further comprises identifying and quantifying the sequenced data (e.g., the sequences of the binding moieties, the barcode sequences, or the primer binding site sequences). For example, the informative patterns can be based on the spatially decoded bounded k-mers if the probes are oligonucleotide probes or decoded chemical moieties if the probes are non-nucleic acid probes.

For analysis of the probe comprising a non-nucleic acid binding moiety (for examples, probes comprising binding moieties composed of peptide or oligourethane), the step of identifying and quantifying the sequenced data comprises extracting the sequences of the barcodes from the sequenced amplicons, determining the patterns in the sequences, and determining the target that bound by the barcoded probes. In some embodiments, the step of identifying and quantifying the sequenced data comprises extracting sequences of the primer binding sites from the sequenced amplicons. In some embodiments, the method further comprises determining the peptide sequences of the binding moieties of the bounded probes, comparing the patterns of the peptide sequences of the bounded probes to the proteome of a protein or a peptide. In some embodiments, the method disclosed herein further comprises translating the quantitative results into a graphic pattern (for examples, as shown in FIG. 8).

In some embodiments, the method further comprises processing the qualified sequencing data using a dimensionality reduction algorithm (e.g., principal component analysis (PCA) or t-distributed stochastic neighbor embedding (t-SNE)).

For analysis of the probe comprising a polynucleotide binding moiety, the step of identifying and quantifying the sequenced data comprises size filtering the reads for the appropriate amplicon size (e.g., 50-500 bp, 50-100 bp. 100-400 bp. 100-300 bp, or 150-200 bp). In some embodiments, the ligated oligonucleotide probes are 180 bp and the probe-only samples are 90 bp. Accordingly, in some embodiments, the step of identifying and quantifying the sequenced data comprises size filtering the reads for amplicon size that is about 180 bp or 90 bp.

Identifying and quantifying the sequenced data further comprises extracting the binding moiety sequences (i.e., the n15 sequences) from the sequenced amplicons, determining a plurality of k-mers of each binding moiety sequence, and determining the counts of each k-mer. In some embodiments, the value of k-mer is set as any of k=3 through k=15. In some embodiments, the value of k-mer is set as k=3, k=4, k=5, k=6, k=7, k=8, k=9, k=10, k=11, k=12, k=13, k=14, or k=15. In some embodiments, the value of k-mer is set as k=8. The p value of a given k-mer can be then calculated. In some examples, the significance of a k-mer's enrichment across all replicates in a foreground (FG) set (e.g., samples containing an analyte or a target of detection) versus a reference control and the individual counts are gathered for both the FG and the control. An analysis method (e.g., the Mann-Whitney U test) is then used for determining the significance of the difference between the distributions in the FG and the control. It should be understood that the reference control can be a probe-only sample or a sample containing a different analyte. For probe-only sample, a sequence dataset can be obtained by directly sequencing the unligated oligonucleotide probe sets separately. In some embodiments, the reference control can be in silico randomly generated sequenced data.

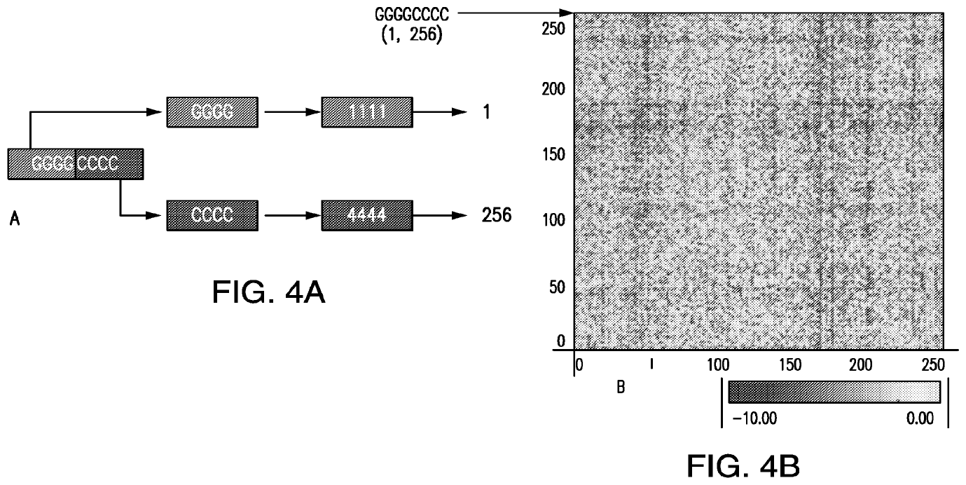

In some embodiments, the method disclosed herein further comprises translating the quantitative results into a graphic pattern. In some embodiments, the method disclosed herein further comprises translating the k-mer counts and the p values thereof into a graphic pattern thereby generating a graph representation of the tested sample. For even values of k, the left and right halves of a k-mer string are encoded as ordered values by assigning a number to each base (e.g., as shown in FIGS. 4A-4B). Accordingly, the method comprises converting a k-mer to a pair of coordinates for plotting a heatmap. The color of said k-mer in the heatmap correlates to the calculated significance (e.g., p value) of said k-mer using the algorithm described herein. The generated graphic pattern can be compared to a reference control (e.g., the pattern of a known substrate). It should also be understood that this algorithm can be trained with binding patterns of known substrates such that patterns in datasets from unknown samples can then be modeled.

Accordingly, in some aspects, disclosed herein is a method of detecting a target in a sample, said method comprising:
obtaining a first oligonucleotide probe and/or a second oligonucleotide probe, wherein said first oligonucleotide probe comprises a 5′ priming binding site, a binding moiety, and a 3′ splinting site, wherein the said second oligonucleotide probe comprises a 5′ splinting site, a binding moiety, and a 3′ priming binding site;
mixing at least one first oligonucleotide probe and/or at least one second oligonucleotide probe with the sample thereby allowing binding of oligonucleotide probe through its binding moiety to a substrate on the target;
ligating the 3′ splinting site of the first oligonucleotide probe and the 5′ splinting site of the second oligonucleotide probe thereby producing a ligated oligonucleotide probe template;
performing amplification of the ligated oligonucleotide probe template thereby producing a plurality of ligated oligonucleotide amplicons;
sequencing the plurality of ligated oligonucleotide amplicons;
extracting the binding moiety sequences (e.g., the n15 sequences) from the sequenced amplicons;
determining a plurality of k-mers of each binding moiety sequence;
determining the counts of each k-mer;
calculating the p-value by comparing the frequency of each k-mer in the sample with a reference control;
translating the counts of each k-mer and the p-value of each k-mer into a graphic pattern thereby generating a graphic representation of the sample; and
comparing the graphical pattern to a reference control (e.g., the pattern of a known substrate) thereby identifying the target.

In some embodiments, the method further comprises processing the qualified sequencing data using a dimensionality reduction algorithm (e.g., principal component analysis (PCA) or t-distributed stochastic neighbor embedding (t-SNE)).

In some embodiments, the target is biotic or abiotic. In some embodiments, the target is a cell. In some embodiments, the cell is a eukaryotic cell or a prokaryotic cell. In some embodiments, cell is a cell of a pathogen, a cell infected with a pathogen, a diseased cell, or a cancer cell.

In some embodiments, the target comprises a viral protein. In some embodiments, the viral protein is a protein of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

103. In some embodiments, the target comprises a bacterial protein. In some embodiments, the bacterial protein is a protein of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, or other Yersinia species, or Mycoplasma species.

In some embodiments, the target comprises a protein of a parasite. In some embodiments, the parasite is Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, or Entamoeba histolytica.

Also disclosed herein is a method of detecting a biotic substrate in a sample, said method comprising:
obtaining a first library of probes and/or a second library of probes, wherein each probe of the first library comprises a 5′ primer binding site, a binding moiety, and a 3′ splinting site, and wherein each probe of the second library comprises a 5′ splinting site, a binding moiety, and a 3′ primer binding site; and
mixing the probes of the first library and/or the probes of the second library with the sample thereby allowing binding of the oligonucleotide probes through their binding moieties to one or more substrates in the sample.

In some embodiments, the method further comprises
ligating the 3′ splinting site of the probe of the first library and the 5′ splinting site of the probe of the second library thereby producing a ligated probe template; and
performing amplification of the ligated probe template thereby producing a plurality of amplicons;
sequencing the plurality of amplicons;
identifying and quantifying the sequenced data;
translating the quantitative results into a graphic pattern; and
determining that the sample has biotic substrate if the graphical pattern of the sample comprises a pattern of a biotic substrate.

Also disclosed herein is a method of diagnosing a cancer in a subject, said method comprising:
obtaining a biological sample from the subject;
obtaining a first library of probes and/or a second library of probes, wherein each probe of the first library comprises a 5′ primer binding site, a binding moiety, and a 3′ splinting site, and wherein each probe of the second library comprises a 5′ splinting site, a binding moiety, and a 3′ primer binding site;
mixing the first library of probes and/or the second library of probes with the biological sample thereby allowing binding of the probes through their binding moieties to one or more substrates in the biological sample;
ligating the 3′ splinting site of the probe of the first library and the 5′ splinting site of the probe of the second library thereby producing a ligated probe template;
performing amplification of the ligated probe template thereby producing a plurality of amplicons;
sequencing the plurality of amplicons;
identifying and quantifying the sequenced data;
translating the quantitative results into a graphic pattern; and
determining that the subject has cancer if the graphical pattern comprises a pattern of a cancer biomarker.

In some embodiments, identifying and quantifying the sequenced data comprises extracting the sequences (e.g., the binding moiety sequence or the barcode sequences) from the sequenced amplicons.

In some embodiments, the method of any preceding aspect further comprises
determining a plurality of k-mers of each binding moiety sequence;
determining the counts of each k-mer.
calculating the p-value by comparing the frequency of each k-mer in the sample with a reference control.

The p-value can be calculated using binomial distribution. In some embodiments, the graphic pattern represents the p-value of a k-mer and the sequence information of the k-mer.

Accordingly, in some aspects, disclosed herein is a method of diagnosing a cancer in a subject, said method comprising:
obtaining a biological sample from the subject;
obtaining a first library of oligonucleotide probes and/or a second library of oligonucleotide probes, wherein the oligonucleotide probe of the first library comprises a 5′ primer binding site, a binding moiety, and a 3′ splinting site, wherein the oligonucleotide probe of the second library comprises a 5′ splinting site, a binding moiety, and a 3′ primer binding site;
mixing the oligonucleotide probes of the first library and/or the oligonucleotide probes of the second library with the biological sample thereby allowing binding of oligonucleotide probes through their binding moieties to one or more substrates in the biological sample;
ligating the 3′ splinting site of the oligonucleotide probe of the first library and the 5′ splinting site of the oligonucleotide probe of the second library thereby producing a ligated oligonucleotide probe template;
performing amplification of the ligated oligonucleotide probe template thereby producing a plurality of ligated oligonucleotide amplicons;
sequencing the plurality of ligated oligonucleotide amplicons;
determining a plurality of k-mers of each binding moiety sequence;
determining the counts of each k-mer;
calculating the p-value by comparing the frequency of each k-mer in the sample with a reference control;
translating the counts of each k-mer and the p-value of each k-mer into a graphic pattern thereby generating a graphic representation of the sample; and
determining that the subject has cancer if the graphical pattern comprises a pattern of a cancer biomarker.

In some embodiments, the method further comprises processing the qualified sequencing data using a dimensionality reduction algorithm (e.g., principal component analysis (PCA) or t-distributed stochastic neighbor embedding (t-SNE)).

In some embodiments, the method disclosed herein of diagnosing cancer further comprises initiating a cancer therapy if the subject is diagnosed as having cancer. A cancer can be selected from, but is not limited to, a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In another aspect, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow; brain, lung, breast, pancreas, liver, head and neck, skin, reproductive tract, prostate, colon, liver, kidney, intraperitoneum, bone, joint, and eye.

It is intended herein that the disclosed methods of inhibiting, reducing, and/or preventing cancer metastasis and/or recurrence can comprise the administration of any anti-cancer agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate), Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab).

The method disclosed herein can determine spatial position of one or more substate on a surface based on the data generated by the PLA assay. Accordingly, disclosed herein is a method of determining spatial position of one or more substrates on a surface, said method comprising:
obtaining a first library of probes and/or a second library of probes, wherein each probe of the first library comprises a 5′ primer binding site, a binding moiety, and a 3′ splinting site, and wherein each probe of the second library comprises a 5′ splinting site, a binding moiety, and a 3′ primer binding site;
applying the first library of probes and/or the second library of probes to the surface thereby allowing binding of the probes through their binding moieties to one or more substrates on the surface;
ligating the 3′ splinting site of the probe of the first library and the 5′ splinting site of the probe of the second library thereby producing a ligated probe template;
performing amplification of the ligated probe template thereby producing a plurality of amplicons;
sequencing the plurality of amplicons;
identifying and quantifying the sequenced data; and
determining the spatial position of the one or more substrates.

Also disclosed herein is a kit comprising a first oligonucleotide probe, a second oligonucleotide probe, and a splint oligonucleotide: wherein said first oligonucleotide probe comprises a 5′ primer binding site, a binding moiety, and a 3′ splinting site; wherein the said second oligonucleotide probe comprises a 5′ splinting site, a binding moiety, and a 3′ primer binding site; and wherein said oligonucleotide splint comprises a first region and a second region, and wherein the first region is complementary to the 3′ splinting site of the first oligonucleotide probe and the second region is complementary to the 5′ splinting site of the second oligonucleotide probe.

### VII. EXAMPLE

### Example 1. Agnostic Analytical Methods for Characterizing Materials

### A. Development of a PLA-Based Assay for Materials Characterization.

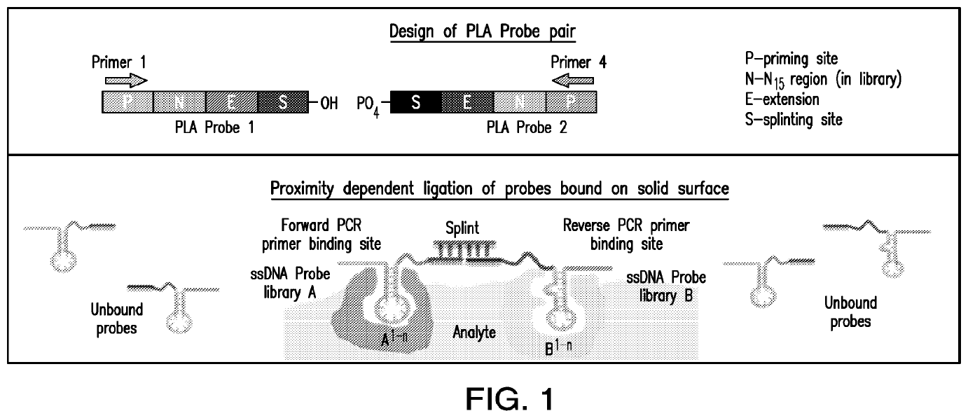

This study develops a PLA-based assay for materials characterization. Libraries were synthesized for ligation and amplification, and the use of the libraries was proofed with a series of materials. Proximity ligation assay (PLA) has been optimized to ‘capture’ pairs of DNA sequences that coordinately interact with a material (FIG. 1). By ligating in place followed by PCR amplification and NextGen sequencing of ligated amplicons, developed herein is a record of transient interactions of single stranded DNA probes on a material. Initially, using sequence-defined PLA ‘probes’ tagged with biotin and both solution phase and immobilized specific (streptavidin) and non-specific (albumin) targets, this study had optimized several PLA parameters including priming sequences, probe length, DNA ligase type and amount, target vs probe concentration, splint length, ligation time and temperature, and PCR conditions to maximize specific signal and minimize background amplicons. When measured using qPCR assays, the optimized protocol yielded a 500-fold enrichment of streptavidin-specific PLA signal versus background in albumin-containing reactions using solution phase targets. With target materials immobilized on magnetic beads, which allowed washing and removal of unbound PLA probes, specific PLA products can be enriched a million-fold above background in qPCR assays. Following development of the optimized PLA protocol, two PLA probe libraries were prepared, each with a 15-mer region of random bases and their ability to be ligated and PCR amplified was confirmed. Subsequently, the two PLA probe libraries were used to perform surface-based PLA analysis of eight different solid materials: (1) Magnetic beads coated with streptavidin (Mag SA), (2) Magnetic beads coated with Chitin (Mag chitin), (3) Magnetic beads coated with Protein A (Mag PrA), (4) 3 mm disc of cellulose paper (Cellulose), (5) 3 mm disc of polyethersulfone paper (PES), (6) 3 mm disc of dark nitrocellulose paper (NC-D), (7) 3 mm disc of PTR5 glass fiber conjugate paper (PTR5), and (8) 3 mm disc of 693 glass fiber paper (693) (FIG. 1). The PCR amplicons resulting from the first seven materials were gel purified and portions were TA cloned into a plasmid and verified by Sanger sequencing of 12 clones from each sample. Most sequenced clones contained a single 180 bp insert of the expected PLA product resulting from ligation of the two probe libraries. Following this confirmation that PLA products were being produced, the remaining portion of five samples, Mag SA, Mag chitin, Mag PrA, PES, and PTR5, were subjected to NextGen sequencing on the MiSeq platform with two million reads per sample. Analysis of this first set of NextGen sequencing data had confirmed that PLA products were generated. Then the next step was to generate several replicate data sets for the analytical pipeline.

Several repeat PLA analyses have been conducted of the same five solid targets—(1) (Mag SA, (2) Mag chitin, (3) Mag PrA, (4) PES, and (5) PTR5, MiSeq data have been collected from these repeats to build the analytical pipeline (see Section B).

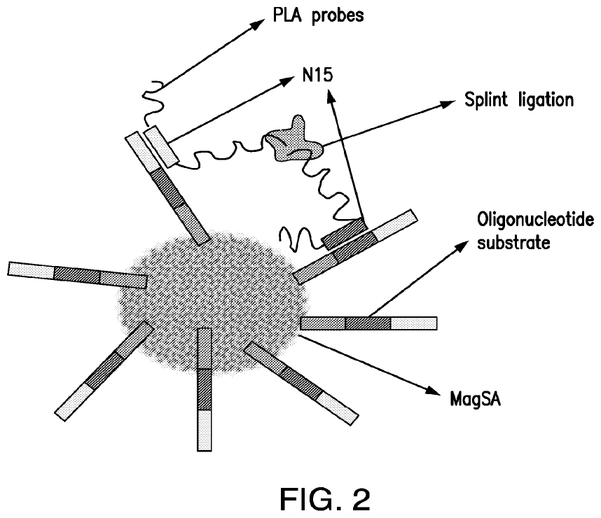

This study has also considered other substrate systems to validate correctness of the PLA methodology. One of these validation substrates include a single stranded DNA with no complementarity to the constant regions of the PLA probes as a proximity substrate. If this PLA protocol is working correctly then the N15 regions of the PLA products enriched using the oligonucleotide as the proximity substrate can show enrichment of kmers with complementarity to the substrate oligonucleotide (FIG. 2). For this first iteration of this validation control, a biotinylated 40-mer oligonucleotide coated on Mag SA beads was used as the substrate. Mag SA beads that were not coated with the 40-mer were used as control. Both types of beads were incubated with the PLA probe libraries and then unbound probes were removed by washing the beads thrice. The remaining probes were ligated and PCR amplified prior to NextGen sequencing. Five replicates of such paired (with and without oligonucleotide substrate) PLA samples are sequenced and the resulting datasets are analyzed using the pipeline described in Section B. Outcomes from this sequencing dataset show further method development and validation for which this study tests additional oligonucleotide proximity substrates of different lengths and sequences as well as vary the number of wash steps performed for removal of unbound probes prior to ligation and PCR amplification.

A second set of validation substrates were selected for PLA analysis that include five non-magnetic beads of uniform 1 um size and controlled surface chemistry. These beads include—(1) polystyrene (PS), (2) PS coated with streptavidin, (3) PS coated with protein A, (4) silica, and (5) silica coated with streptavidin. Protocol optimizations are developed to perform PLA analysis and sequencing using these substrates. This study applies the protocol developed using magnetic beads except that any necessary separation of these non-magnetic beads from the aqueous phase is performed using centrifugation.

### B. Development of an Informatics Pipeline to Achieve Differentiation Between PLA Amplification Products.

The deep NGS data generated via sequencing of ligated and amplified DNAs potentially provides a readout or fingerprint of the materials the libraries were in contact with. In order to best understand how to read this fingerprint, this study develops an analytical pipeline that involves a variety of statistical methods.

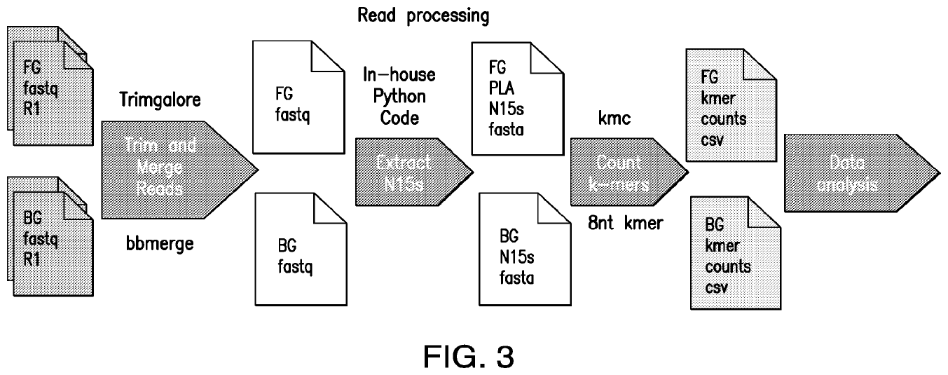

NGS Pipeline. For each sample run on the MiSeq platform, two .fastq files containing the read data were received, one for each set of the paired-end reads. Each of the files were run through a Snakemake pipeline developed by the Ellington Lab to extract the N15 variable regions from the sequences. An overview of the extraction pipeline is shown in FIG. 3. To assess the quality of the reads, each of the .fastq files were analyzed using FastQC. The overall quality of the samples were satisfactory with an expected drop in quality at the 3′ end of the reads. Next, adapter sequences were trimmed from all reads using Trimgalore. The trimmed paired-end read files were merged using PEAR which allows for some error-correcting where the paired-end reads overlap. FastQC was run again on the merged reads and showed high Q scores over the entire 180 bp PLA product and over the 90 bp sequences for the probe-only samples. Next, the N15 regions were extracted from the sequences using a custom Python script. Fuzzy matching using regular expressions was employed to locate each of the N15s. For the extraction, a mismatch threshold for each of the sequences flanking the N15s was set at 10%; sequences with too many mismatches or with insertions/deletions were discarded from further processing. The extracted N15s were output to a FASTA file for downstream analysis. For samples with an analyte present, approximately 73% of sequences on average were PLA sequences of ligated probes with a range from 59% to 79%. For probe-only samples, 85% of sequences on average were unligated probes with a range from 83% to 86%. See Table 1. The FASTA files containing the N15 sequences were then passed to kmc to count k-mers for k=3 through k=15. Each sample's k-mer counts were output as a text file. After all samples were processed, the k-mer files for the same value of k were concatenated into a single .csv file [Kmers.csv] for downstream analysis; there is one file per value of k containing the k-mer counts for all samples. Before performing any statistical analysis, the k-mer counts are normalized by dividing the count by the total number of N15 sequences extracted from the sample and multiplying by 1,000,000.

Analysis Pipeline. Currently, two approaches are used to compare relative k-mer enrichment between a pair of analytes. For single samples, the Kmers.csv file is used to create a 2×2 contingency table and Fisher's exact test is used to calculate the p-value for a given k-mer. To calculate the significance of a k-mer's enrichment across all replicates in a foreground (FG) set (samples containing an analyte) versus a background (BG)/control set (probe-only samples or samples containing a different analyte), the individual counts are gathered for both the BG and the FG from Kmers.csv and the Mann-Whitney U test is used for determining the significance of the difference between the two count distributions. Until a larger number of replicates across all samples was created, the highly skewed counts from the exponential amplification process and small sample sizes prohibit us from using the Central Limit Theorem and its corresponding parametric tests for sample means. However, a simulation pipeline is developed to supplement the statistical analysis. By using the nucleotide frequencies from the processedNGS data, additional data from a BG sample can be simulated under the null assumption that enrichment of individual sequences is independent of the interactions between the probes and the analyte in the FG sample. For each simulated sample, the same number of N15s are generated as extracted from the FG using the BG nucleotide frequencies. Sets of N15s can be repeatedly generated to yield a distribution of k-mers counts that can be used determine the probabilities of different count values. The simulation pipeline is developed to supplement the experimental data.

TABLE 1
N15 extraction efficiencies for all samples.

| sample | PLA_product | unligated_probes | Discarded | total |
|---|---|---|---|---|
| JA20063_MagChitin_S1 | 78.98% | 14.25% | 6.77% | 1106874 |
| JA20063_MagProteinA_S2 | 78.20% | 14.31% | 7.49% | 1450605 |
| JA20063_MagSASS3 | 78.55% | 13.75% | 7.70% | 1901869 |
| JA20063_PES_S4 | 79.45% | 12.11% | 8.44% | 1440551 |
| JA20063_PTR5_S5 | 76.08% | 16.14% | 7.78% | 1477179 |
| JA20198_MagChitin_S1 | 63.77% | 22.28% | 13.96% | 908046 |
| JA20198_MagProteinA_S2 | 70.27% | 19.10% | 10.63% | 974297 |
| JA20198_MagSA_S3 | 74.14% | 18.49% | 7.37% | 1159936 |
| JA20198_PES_S4 | 59.30% | 21.96% | 18.74% | 940646 |
| JA20198_PTR5_S5 | 68.35% | 16.84% | 14.81% | 938320 |
| JA20219_MagChitin_S1 | 75.44% | 15.80% | 8.77% | 1748445 |
| JA20219_MagProteinA_S2 | 73.93% | 15.86% | 10.21% | 1542693 |
| JA20219_MagSA_S3 | 77.98% | 14.31% | 7.71% | 1883083 |
| JA20219_PES_S4 | 73.77% | 14.32% | 11.91% | 1867849 |
| JA20219_PTR5_S5 | 76.38% | 15.51% | 8.11% | 1876930 |
| Probe12-1_S1 | 0.00% | 85.64% | 14.28% | 1964613 |
| Probe12-2_S2 | 0.00% | 85.01% | 14.89% | 1875721 |
| Probe12-3_S3 | 0.00% | 83.00% | 16.88% | 1513337 |
| Probe34-1_S4 | 0.00% | 85.34% | 14.59% | 1499715 |
| Probe34-2_S5 | 0.00% | 85.29% | 14.66% | 1359026 |
| Probe34-3_S6 | 0.00% | 83.66% | 16.29% | 1368230 |

To determine the significance of the complete set of k-mers (a k-mer fingerprint) present in a FG sample compared to a BG, the Mahalnobis distance (MD) is calculated from a FG sample (an k-dimensional vector of k-mer counts) to the distribution of BG samples. Unlike the Euclidean distance, the Mahalanobis distance accounts for the covariance between k-mers. For k <15, the study can have a considerable amount of covariation between individual k-mer counts due to overlap between k-mers during counting. If all of the k-mer counts were distributed normally, the chi-squared distribution can be used to calculate p-values for the MD. Log 2-transformed k-mer counts can be approximately normally distributed. Thus, this study simulates a distribution of MDs from the background to determine the significance of the FG MD. In addition to yielding a significance measure for a k-mer fingerprint, MDs also allow ranking the differences between various FG samples and a chosen BG.

As an aid to this numerical analysis, a method to visualize k-mer fingerprints was developed using heatmaps. For even values of k, the left and right halves of a k-mer string are encoded as ordered values by assigning a number to each base. We used: G->1, A->2, T->3, C->4. Then each of the ordered values is mapped to a number from 1 to 4\hat{ }(k/2). So each k-mer is represented by a pair of coordinates on a 2D-plane. Using this approach, the k-mer heatmap has symmetry over the x-y line. The 2D-plane is divided into equally sized squares for each k-mer and colored according to a measure of interest: normalized counts, fold enrichment (compared to BG), enrichment p-values (compared to BG), etc. See FIG. 4. The heatmaps offer a quick way to find salient patterns across the different analytes.

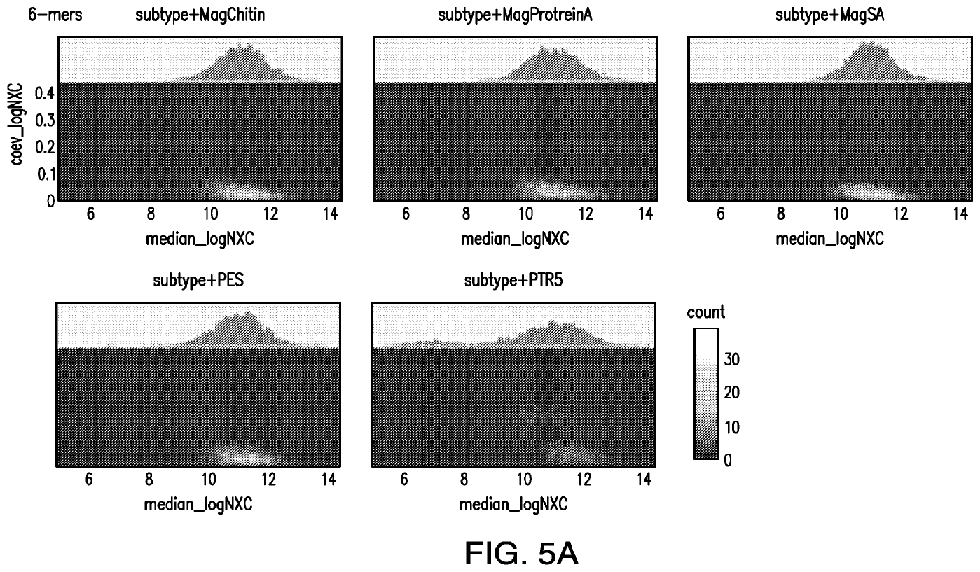

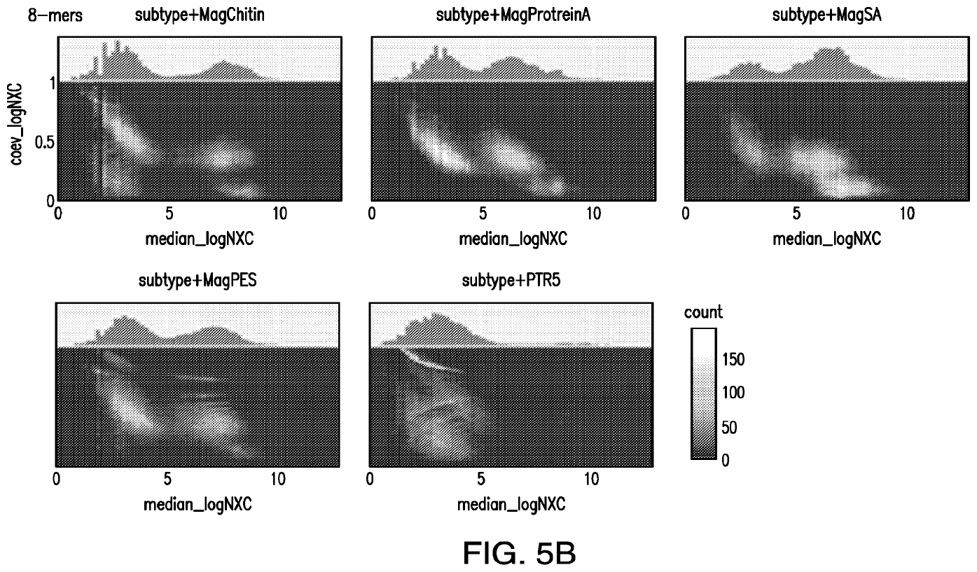

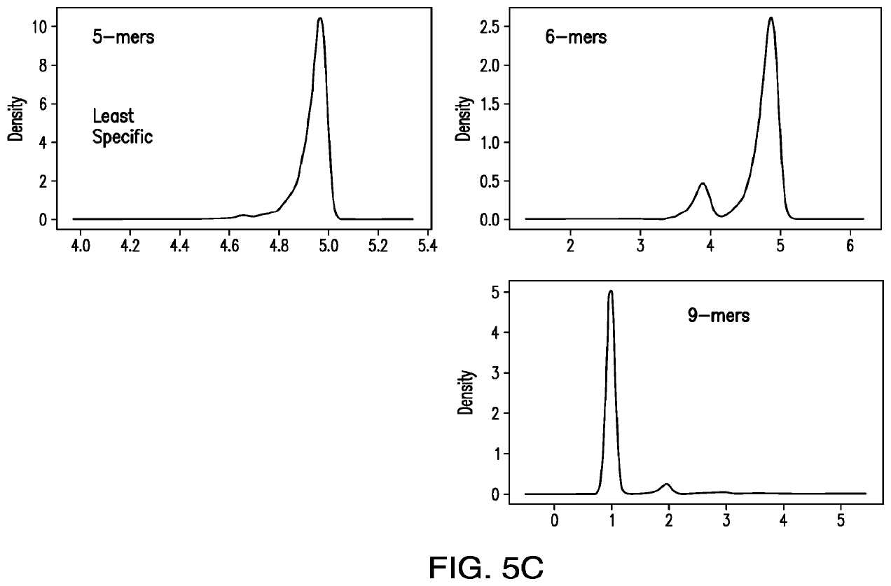

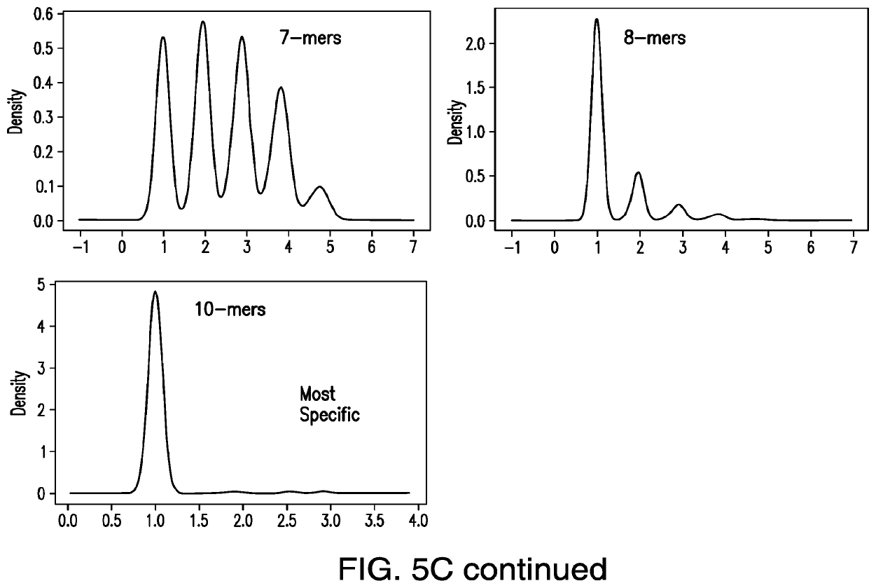

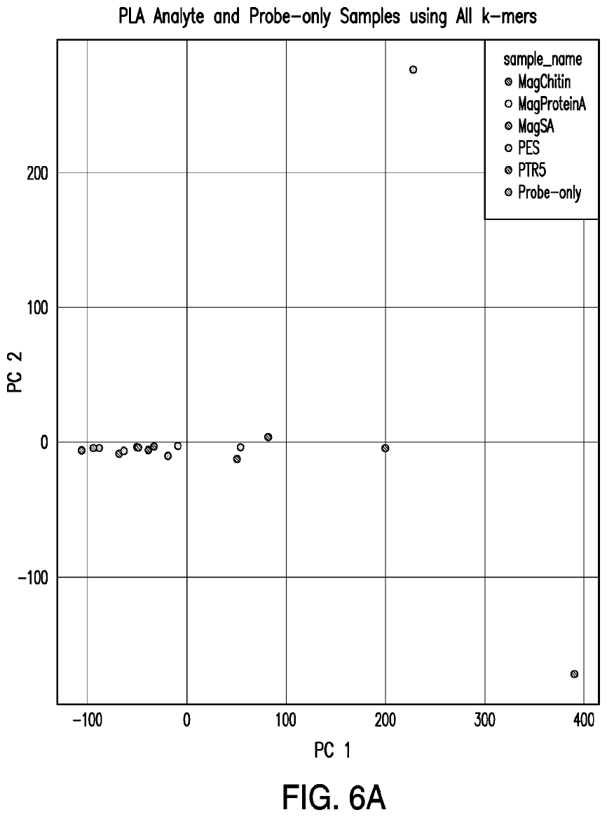

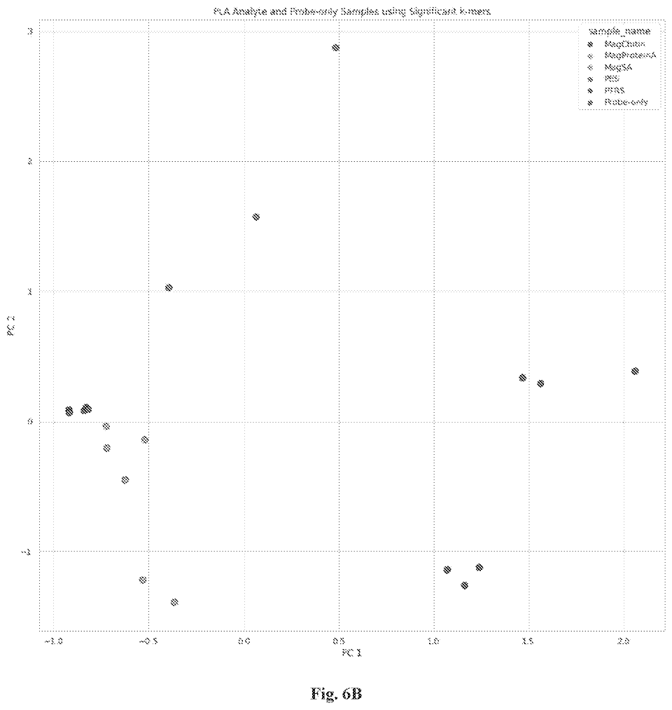

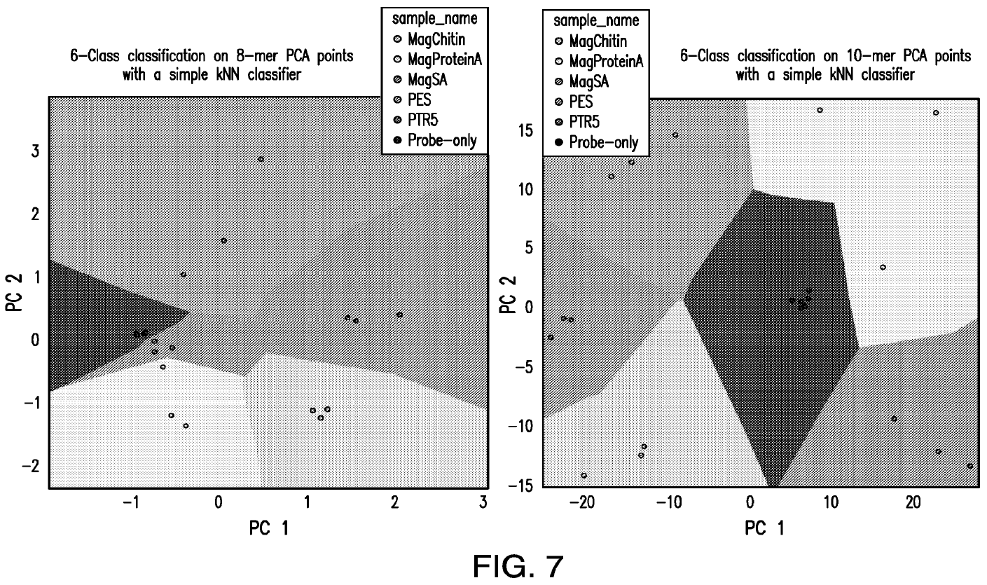

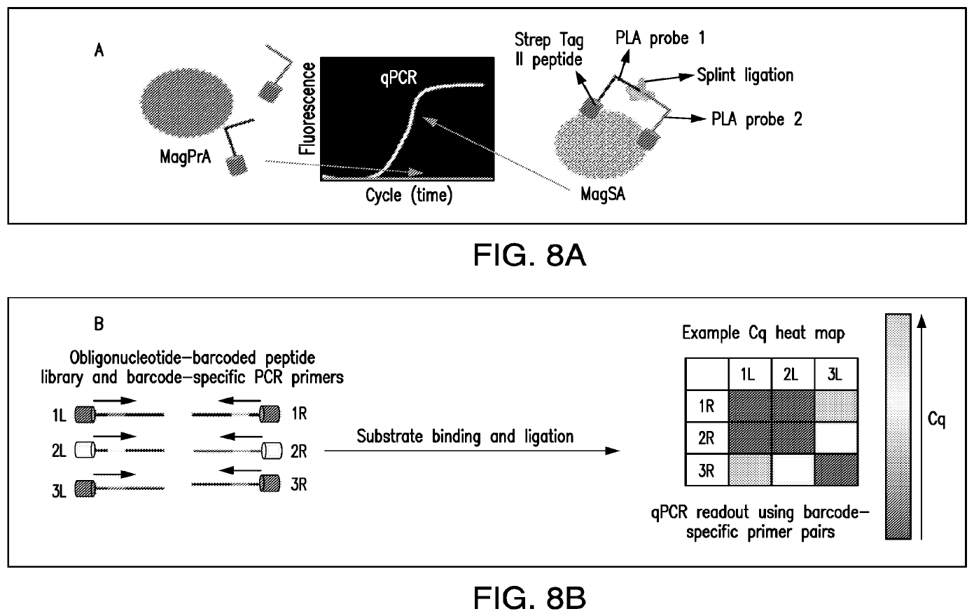

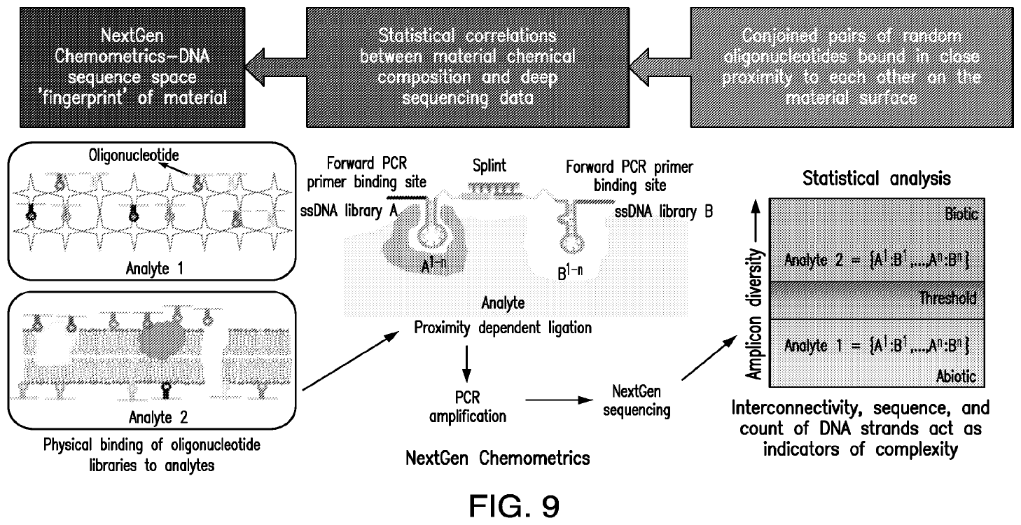

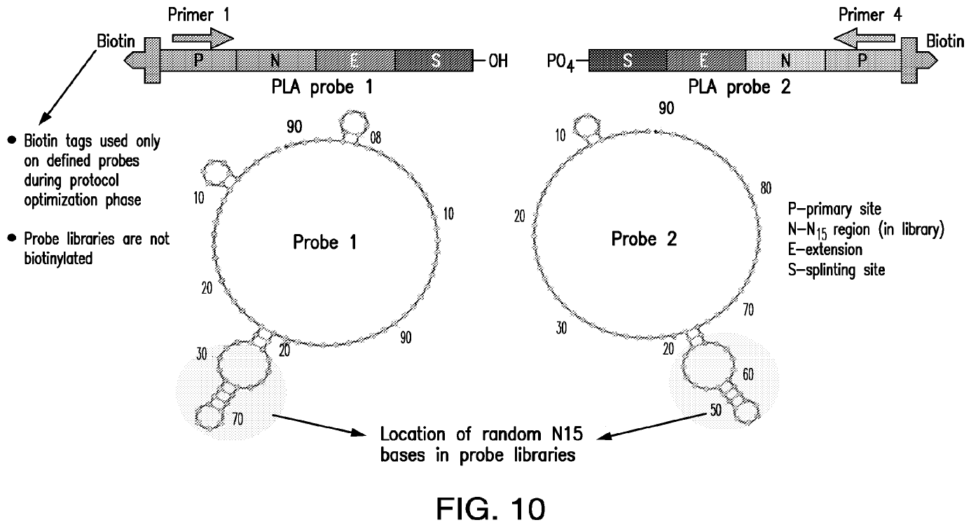

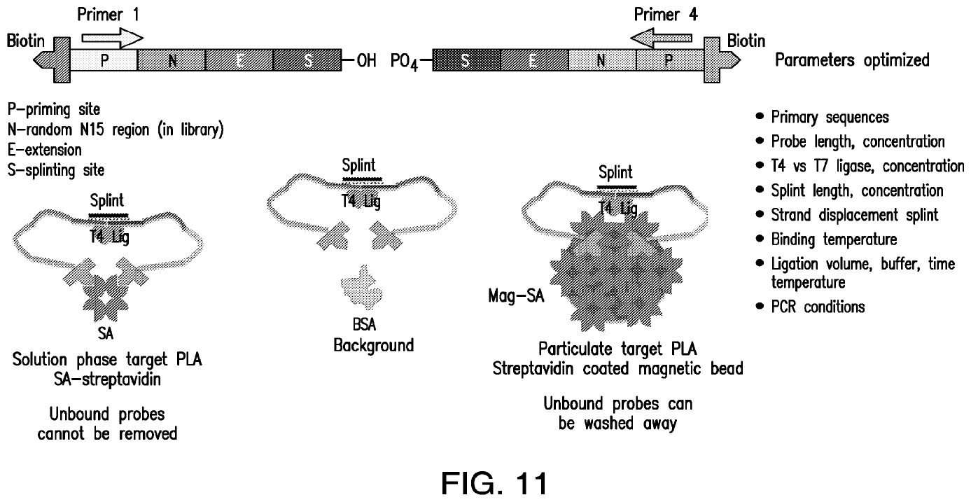

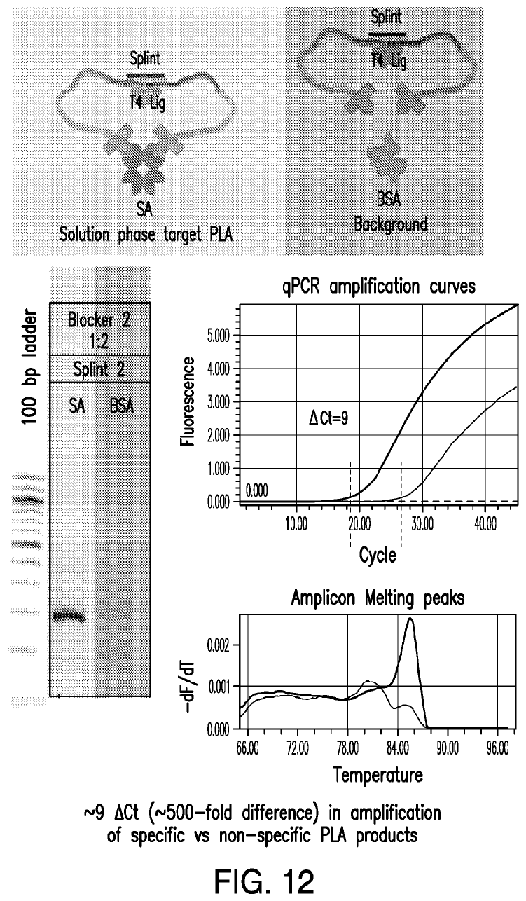

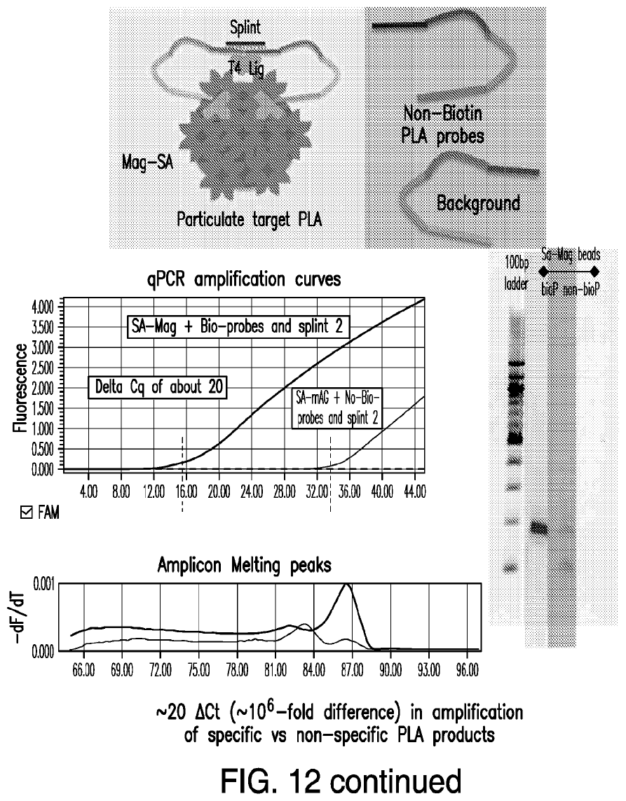

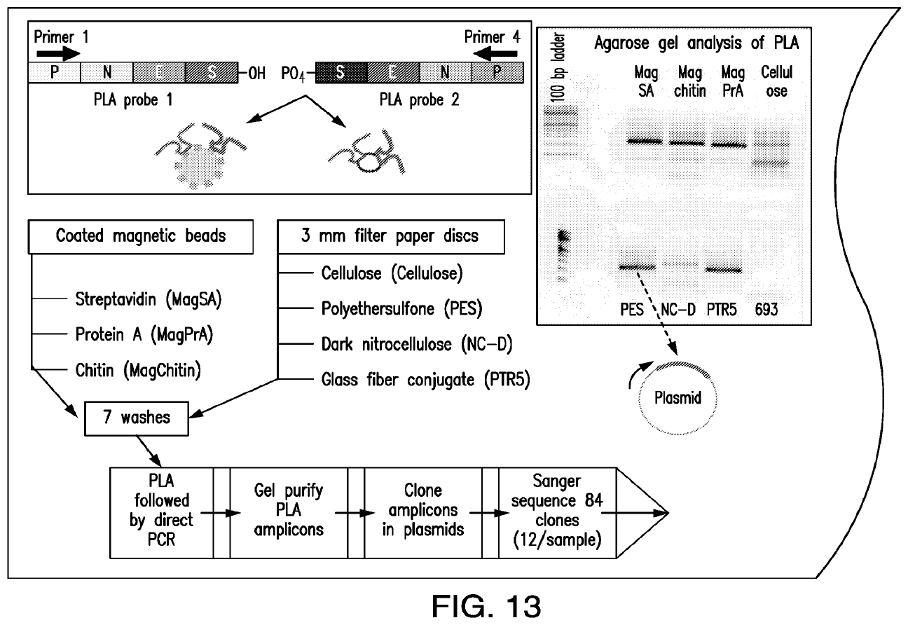

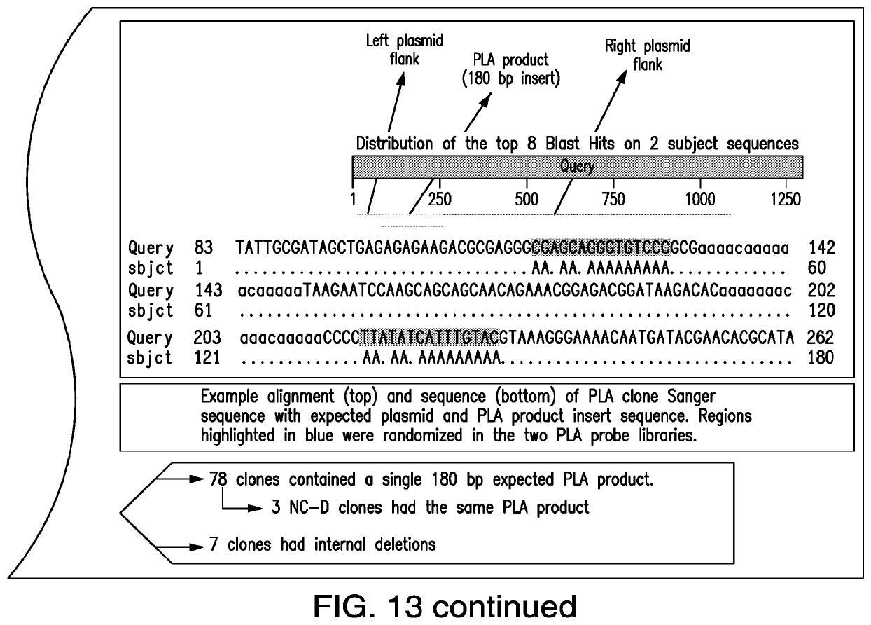

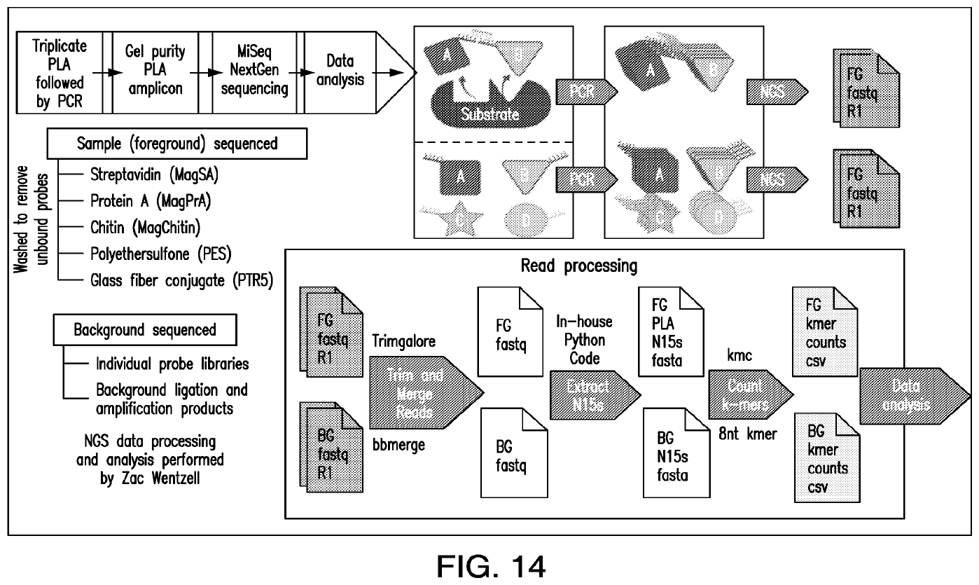

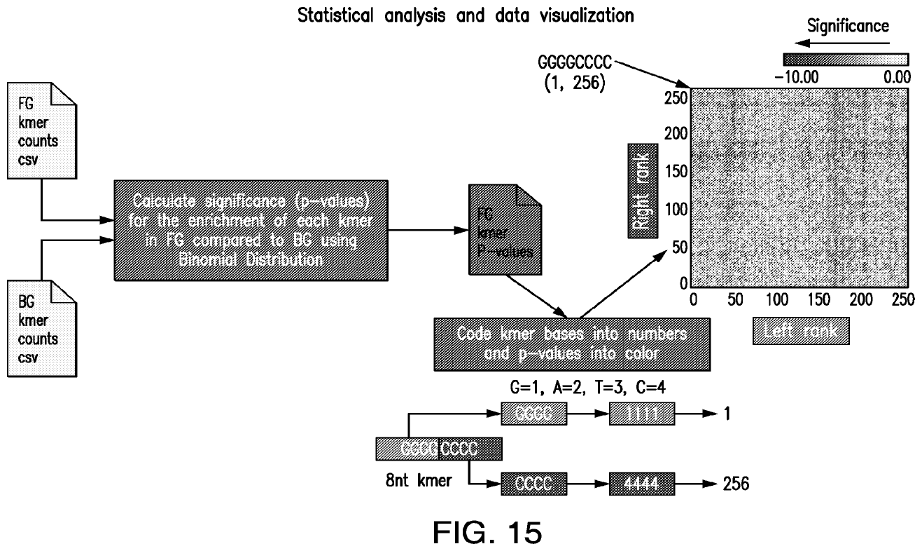

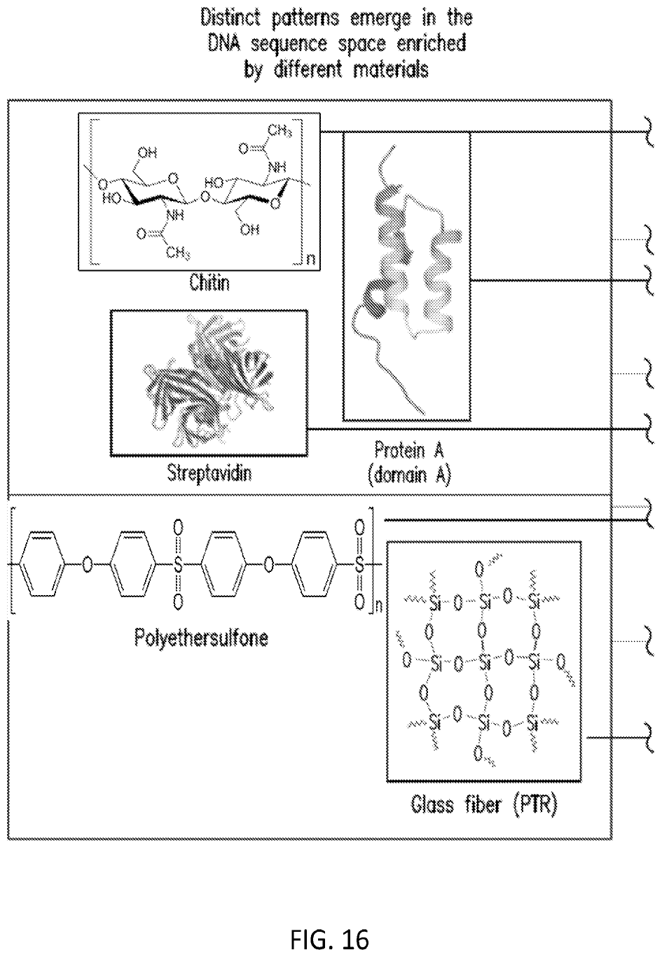

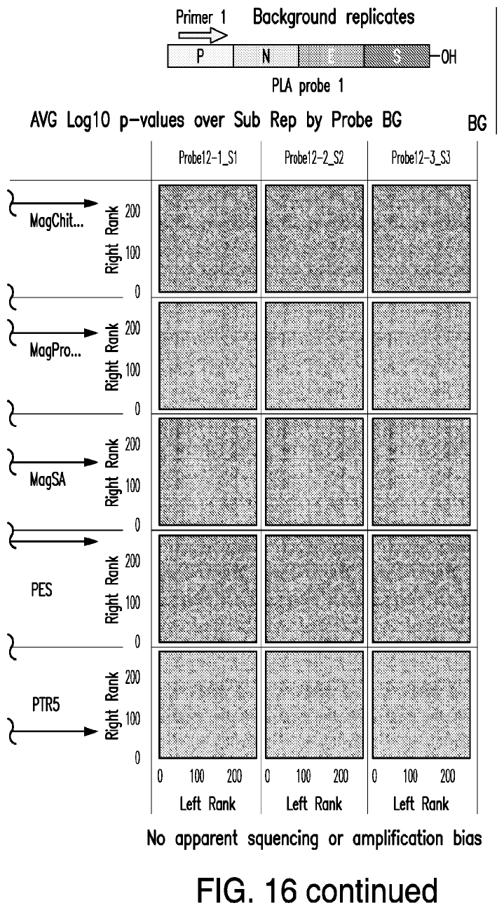

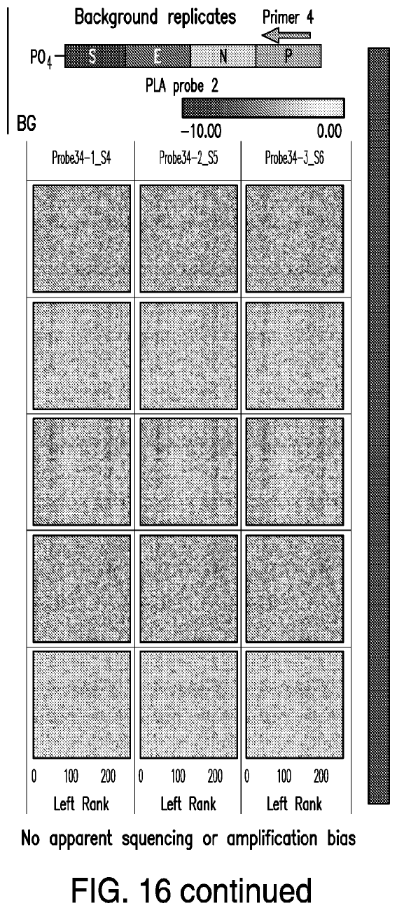

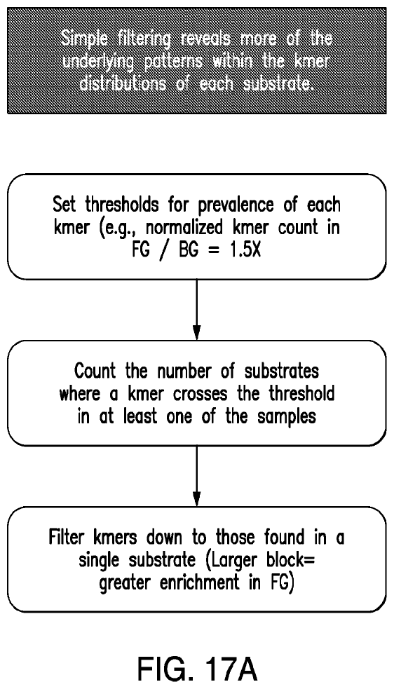

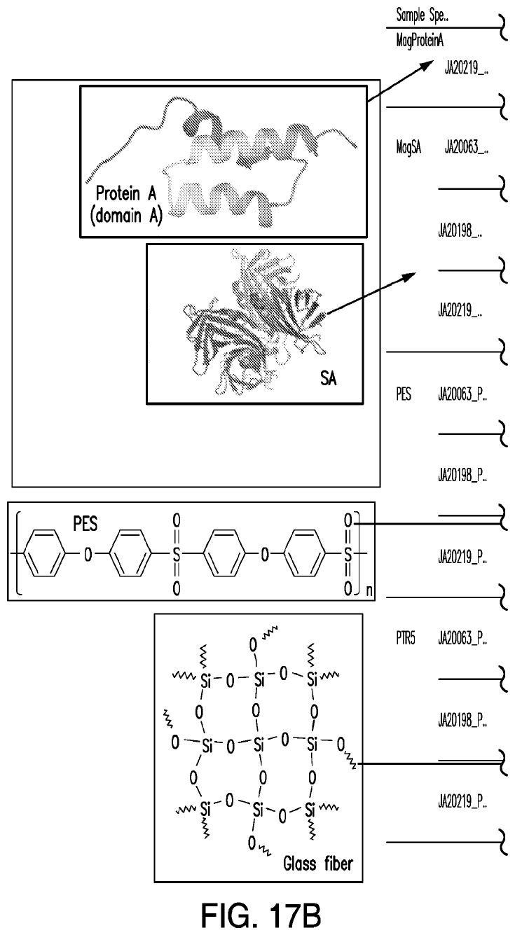

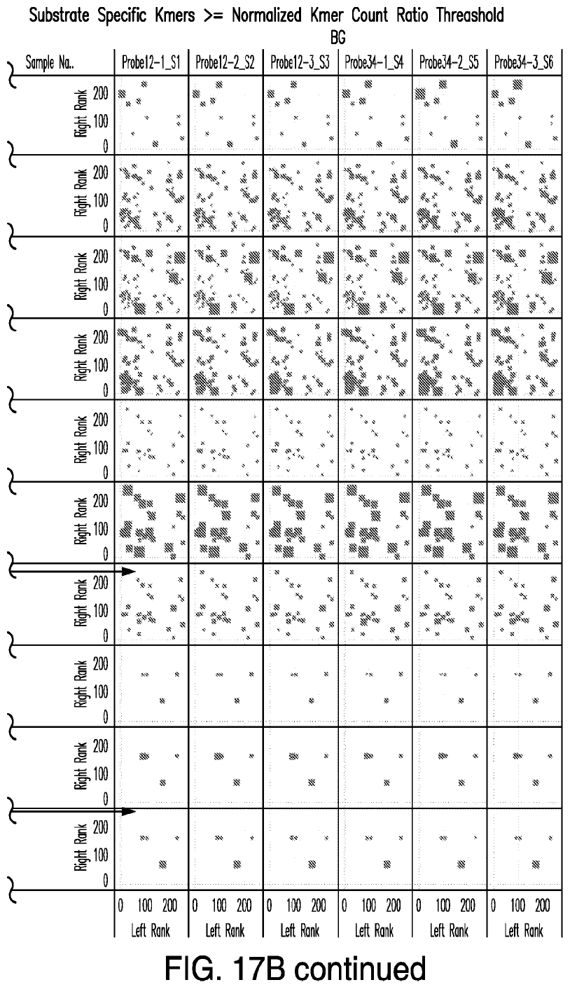

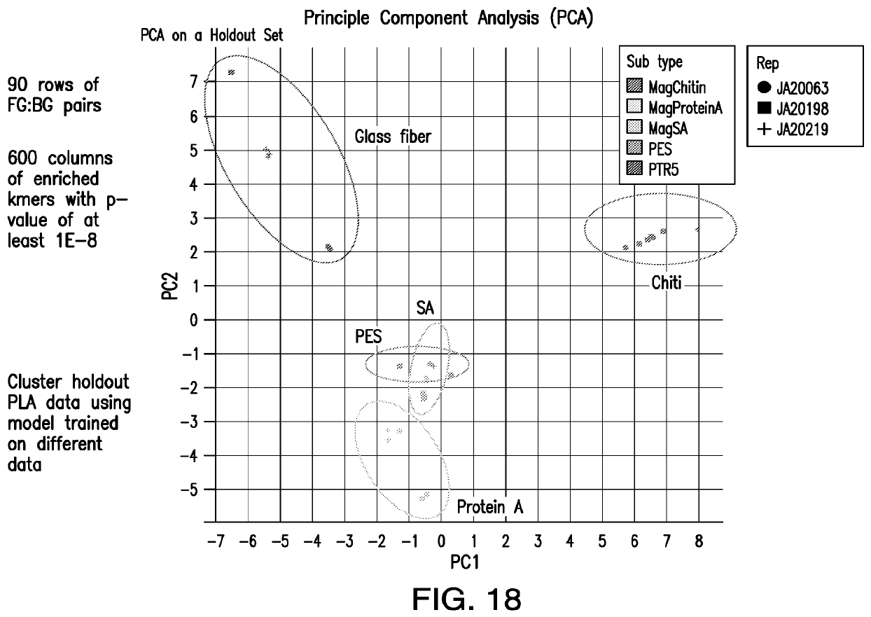

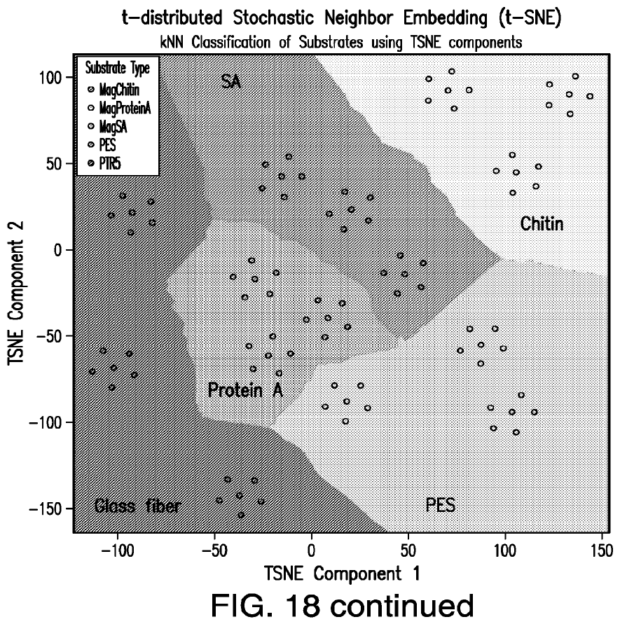

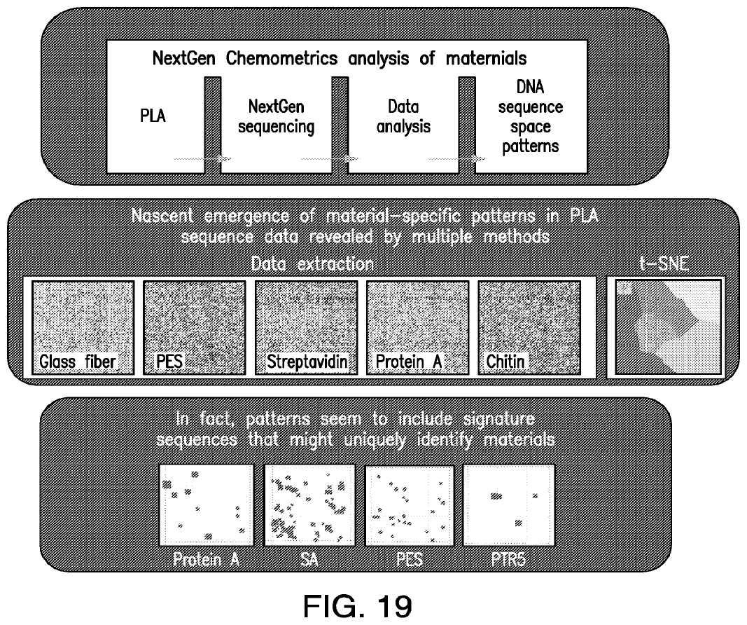

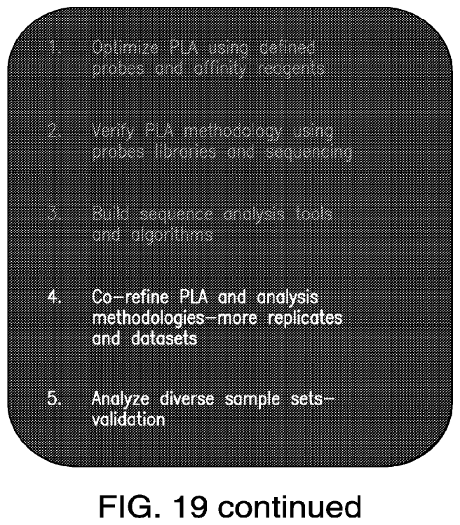

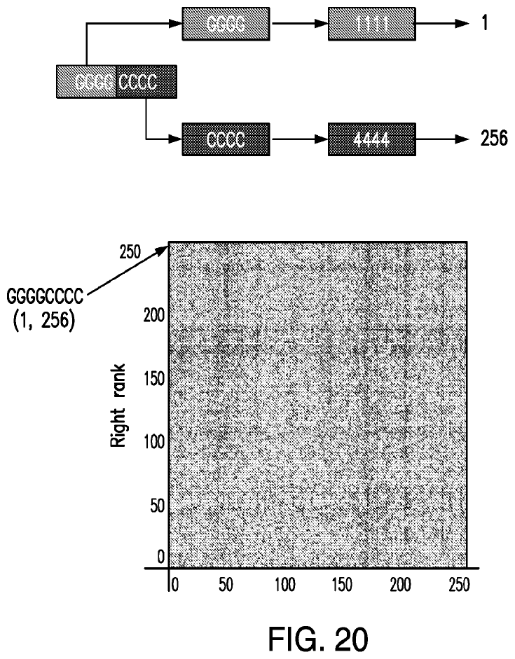

It can be beneficial to choose a subset of sequences for the probe library. For example, if this study identifies sets of sequences that interact with certain macromolecules with high specificity, to the study can train a model to predict the composition of a sample using the fingerprint. Towards this goal, this study has developed a method of identifying and extracting highly specific k-mers. The method begins by calculating the median and coefficient of variation of each k-mer's log 2 counts over all replicates for each analyte. The coefficient of variation (CoV) serves as a measure of enrichment precision over replicates. The median better accounts for large fluctuations over a small number of replicates than the mean. Next, k-mers are filtered to those with a median log 2 enrichment>than an enrichment threshold and a CoV<than a variation threshold value. These thresholds are determined empirically from the distributions of both metrics. See FIG. 5. After an initial set of highly enriched, low variance k-mers have been found, the median k-mer enrichments (over replicates) for each analyte are used to compute the entropy over all analytes for each selected k-mer. Finally, the entropy is used to rank each of the k-mers with a preference for low entropy. To determine a threshold for selecting these low entropy k-mers, the method defines the effective substrate specificity ESS as e(k-mer entropy). ESS values close to one indicate that k-mer enrichment in one analyte outweighs the rest. However, the choice of k effects the average entropy of the k-mers. For smaller values of k, specificity was lost and the overall variance of kmer counts decrease (see FIG. 5). This results in increasing values for ESS making it harder to identify substrate specific k-mers. For higher values of k, specificity increases at the cost of losing sensitivity. Therefore, the ideal value of k may vary depending on the analysis being performed and the surface characteristics of the analytes.

Principle Component Analysis. Principal Component Analysis (PCA) allows the current study to take a high-dimensional dataset (such as k-mer counts for a sample) and map it onto a more informative lower-dimensional space. It serves as a method to reduce noise present in the data and enables the visualization of the data in 3 or fewer dimensions. As an unsupervised learning algorithm, the model never sees which analyte a data point belongs to. But instead, it tries to project the data onto a new set of orthogonal dimensions (as linear combinations of the original dimensions) with maximum variance (information) across all the data used for training. As with most machine learning models, the curse of dimensionality (having more dimensions than training examples) can negatively impact the model. The number of features grows exponentially with this k-mer length. However, as discussed previously, lowering the k-mer length also directly impacts specificity. PCA's focus on variance and covariance can also cause it to overestimate the importance of noisy k-mers. e.g. a set of completely random, noisy k-mers that also covary with each other due to the overlap effect in k-mer counting. These k-mers can be considered valuable by the model, especially for a lower number of data points. To aid the model, this study can reduce the number of k-mers that are used as features by filtering down to a smaller, more informative set. This study can use the enrichment significance or the entropy across samples to prune away noisier k-mers from the dataset before fitting this model. As thenumber of samples increases, this can become less of a concern. As part of the current pipeline, p-values from the k-mer level analysis are used to prefilter the data; specifically, the p-values came from the Mann-Whitney U-test between an analyte's replicate counts for a given k-mer versus the counts from all other samples. When fitting the model, the log 2(count) can be used for each k-mer to remove some of the non-linearity from the features, since PCA is a linear model. FIG. 6 shows a model trained using all 8-mers (left) versus a filtered set of 8-mers(right). A drastic improvement is observed in the quality of the principal components to group these samples by their specific analyte. Based off the plots from FIG. 5, k=10 was the first value of k where almost all highly enriched, low variance k-mers were also substrate-specific. Thus. 10-mer data were ran through the same PCA pipeline described previously to compare the results. 10-mers achieved improved partitioning of the analytes (FIG. 7). For the current data set, higher values of k resulted in lower quality partitioning of the analytes. As more data are gathered, this PCA model can be improved and the model's component loadings can be examined to better understand the similarity and differences between analytes. As a more rigid model, seeing meaningful partitioning of the samples for such a small dataset is promising. The pipeline for the PCA model allows the transformation of new sample onto this 2D space where classification can be performed or inferences can be made on unknown analytes (FIG. 7). As more data are collected, more advanced models can be tested for learning representations of the k-mer fingerprint feature space, such as deep neural networks.

C. Development of Libraries with Affinity Ligands.

While nucleic acids are remarkably adept at folding into structures that can bind specific ligands (e.g., aptamers), the methods described herein are directed towards broader, more semi-specific interactions for global characterization, and thus the chemistries inherent in G, A, T, and C (and the sugar and phosphodiester backbone) can ultimately be limited. To overcome this barrier, oligonucleotides for PLA are conjugated to a set of different chemistries, embodied in individual peptides and individual oligourethanes. In this way, new chemistries can be further transduced into binding and hence into amplicons for NGS analysis.

This study conjugates individual barcoded oligonucleotides to peptides, in order to create a small library of 5′ and 3′ conjugates that can in turn act as peptide affinity reagents to probe surfaces, via PLA. This system allows the uses of peptide libraries, instead of oligonucleotides, as the proximity binding agent (FIG. 8). Meanwhile, short, barcoded oligonucleotides covalently attached to the peptides can serve as the PLA reactants that can undergo ligation followed by PCR amplification when two peptides, bearing these oligonucleotides, bind close to each other on a material. To begin to test and optimize the peptide-PLA system, an 8 residue Strep Tag II peptide was obtained from the Anslyn lab that is functionally similar to biotin in that it binds to Streptavidin. This allows the use of the well characterized streptavidin binding to validate and optimize the peptide PLA methodology. As a first step, a pair of 50-mer single stranded DNA molecules were designed to serve as the PLA reactants. One member of the pair has a 5′-end azide moiety while the other oligonucleotide has a 3′-end azide group and a 5′-end phosphate group. Using the azide groups, each oligonucleotide can be covalently attached to separate aliquots of the Strep Tag II peptide with an alkyne addendum using Click chemistry. The resulting peptide-DNA conjugates were gel purified and peptide PLA protocols were developed using streptavidin-magnetic beads and protein A-magnetic beads as the specific and non-specific substrates and qPCR using barcode-specific primers for readout. Eventually peptide sequences are chosen to span the range of chemistries inherent in the 20 amino acids, which are inherently greater than for the 4 nucleotides we have previously examined. The hope is that even with small libraries of peptide conjugates, the combinatorial power of binding (n peptide conjugates×n peptide conjugates) can provide deep and detailed information on co-adjacent amplicon barcodes. Moreover, since the chemistries of the amino acids and peptides are well understood, information can be immediately garnered on whether a surface is generally polar, charged, hydrophobic, aromatic, and so forth.

This method is clearly extensible to other affinity reagents Whether oligourethanes coupled to barcoded nucleic acids can perform a similar function is examined. As with the original examinations of nucleic acid k-mer space (Section B), these experiments can not only provide insights into the complexity of fingerprints, but also allow a direct comparison between different probes in terms of the extent to which they generate that complexity (or lack thereof). During the execution of these projects, this study can directly compare the number and type of co-adjacent binding species between nucleic acids, peptides, and oligourethanes, and to begin to extend to chimeric libraries, as well (peptide:oligourethane, or nucleic acid:peptide couples).

Development of an agnostic method to characterize materials based on the proximity ligation assay. By ‘transducing’ binding information into ligation, and hence into amplicons, this study generates NGS data for materials characterization. Such NGS data is a generalizable fingerprint, and the use of NGS on spaceflights or in extraterrestrial probes can be readily contemplated.

Development of an analytical pipeline to identify statistically valid fingerprints for materials. By determining statistically significant representations of k-mers based on transduction of materials into NGS data, this study again provide a novel and powerful means of materials characterization, but also provide an entirely new paradigm for thinking about the informational complexity of materials.

Development of additional affinity probes for materials. While nucleic acids are remarkably adept at folding into structures that can bind specific ligands (e.g., aptamers), the methods described herein are directed towards broader, more semi-specific interactions for global characterization, and thus the chemistries inherent in G, A, T, and C (and the sugar and phosphodiester backbone) can ultimately be limited. To overcome this barrier, oligonucleotides for PLA are conjugated to a set of different chemistries, embodied in individual peptides and individual oligourethanes. In this way, this study further transduces new chemistries into binding and hence into amplicons for NGS analysis.

Generation of several replicate NextGen sequencing datasets representing PLA analysis of six substrates (streptavidin-coated magnetic beads, oligonucleotide-coated streptavidin magnetic beads, protein A-coated magnetic beads, chitin-coated magnetic beads, polyethersulfone paper disc, and glass fiber conjugate paper disc).

Development of a data analytical and informatics pipeline for differentiating material-specific patterns in the PLA sequencing data.

Conceptualization of peptide-DNA probes for expanding the chemistry of PLA probe sets and initiation of control experiments using a well-defined peptide.

### Example 2. NextGen Chemometrics Using Proximity Ligation Assay (PLA)

This study shows an illustration of what is expected to see in a washed substrate sample and a probe background. The different shapes represent distinct N15 regions. While the substrate sample may have started with the same set of probes (A. B. C. D) as the background, washing should remove any unbound probes yielding a distinct signature based on the characteristics of the material's surface. PCR can equally amplify any probes present in a sample. NGS offers a snapshot of the final concentrations of all the probes that were present in the form of a 2 fastQ files of reads (R1, R2: because this study performed paired end sequencing). The reads present in each of the fastQ files are then processed to remove any adapter sequences present. The adapter-trimmed paired-end reads are then merged into one higher quality read by aligning the overlapping regions and comparing quality scores at each position, lower quality ends of the reads are also trimmed based on a quality score threshold (20). Each merged read is parsed by a python script that we wrote to extract the N15 region (s, there are 2 of them in ligated products; one in a unligated probe read). The script first determines whether each read is a ligated product or a single unligated probe. Ligated probes are used for the N15 extractions in substrate-containing samples, while unligated probes are used for N15 extraction in the probe background samples. The set of N15s extracted from each sample are then output in a single fastA file. The fastA files are then processed by KMC to quickly count the number of each k-mer present over all of the N15 sequences in each file. Finally, the k-mer counts are output as a csv file (each line representing a single k-mer and it's count in the sample) for downstream data analysis (FIG. 14).

The binomial distribution is used to calculate p-values for the enrichment of each k-mer in the foreground sample compared to the background sample. X˜Binom(N,p) is read X is distributed as a binomial random variable with parameters N and p. When performing a set of N trials/experiments, the binomial distribution calculates the probability of X successes over all of the trials, given that each trial has a probability of success, P. For this experiment, a trial is choosing a k-mer at random from the background distribution of k-mers. The total number of trials (N) is represented by the total number of k-mers present in the foreground sample (the total number of times we chose a k-mer at random from the background). In each trial, the probability of choosing a specific k-mer from the background (p) is given by its relative frequency in the background. The number of successes (X) is the number of times the specific k-mer was chosen from the background over all of the trials performed (again a trial here is picking a k-mer at random from the background sample). The probability of choosing a specific k-mer X # of times is what the binomial distribution calculates for each value of X (FIG. 15).

Shown herein (FIG. 16) is a heatmap of k-mer p-values for each pair of substrate FG and probe BG. The color scale shows blue for high significance (lower bound at −10, pvalue of 1E-10) and orange as low significance (max Log 10 pvalue of 0=>pvalue of 1.0), the boundary between the colors is at a Log 10(pvalue) of −2 [=>pvalue of 0.01] is light grey with a continuous color range over the range of values. Magnetic beads are highlighted in green, while plastic and glass filter paper samples are highlighted in yellow. This image shows that the k-mer fingerprints for each of the materials are fairly consistent across all background replicates. Different materials have different fingerprints. However, since there are over 65K 8-mers, some filtering needs to be applied to make it easier to distinguish unique patterns.

Here some of the noise in the previous heatmap was clearned up by setting thresholds for how prevalent a given k-mer has to be in a substrate. First, an enrichment threshold is determined. The normalized (over the total number of kmers in the sample) kmer count in the FG is at least 1.5× the normalized count in the BG sample. Then for each kmer, a count of the distinct number of substrates is performed where at least one of the samples had a FG count at 1.5× the background. Next, the kmers were filtered down to those that were only found in a single substrate. The size of the blocks that make up the heatmap correspond to the exact kmer count ratio of the given kmer: a larger block means higher enrichment in the FG sample compared to the given BG sample. Even a “simple” filtering such as this is able to reveal more of the underlying patterns within the kmer distributions of each substrate (FIG. 17).

There are three replicates per substrate. By using the probe backgrounds for the FG and BG pairs to generate 8-mer data, this study was able to extend our data set a bit further. In this data, there were 90 rows of substrate FG, probe BG pairs and 600 kmer related columns. The number of kmers was first reduced by capturing more substrate specific kmers with a p-value of at least 1e-8. Both PCA and t-SNE (tee-snee) were able to partition the data into meaningful clusters. This a good indicator that there is useful amount of signal present in this PLA data.

The left panel of FIG. 18 shows that a hold out set of PLA data were transformed using a PCA model trained on different data. This demonstrates that the principal components learned from one set can be used with new data the model has not seen before.

The right panel of FIG. 18 shows that this study passed the same data set to a t-SNE model to perform low dimensional clustering. Being less rigid than the linear PCA model, t-SNE was able to cluster the substrate exceptionally well. This study can also use machine learning approaches such as these to identify what substrates are present in a sample.

The Binomial Distribution is used to calculate p-values for the enrichment of each k-mer in the Foreground (substrate-containing) sample compared to the Background sample. The null hypothesis is the FG and BG have the same underlying k-mer distribution. Let X be the count of a specific k-mer in the FG.

$$X ∼ Binom ( N , p ) where N = {\sum }_{i} \vert {kmer}_{i} in FG \vert , p = \frac{\vert {kmer}_{i} in background \vert}{{\sum }_{i} \vert {kmer}_{i} in background \vert}$$

X˜Binom(N,p) is read X is distributed as a binomial random variable with parameters N and p.

When performing a set of N trials/experiments, the binomial distribution calculates the probability of X successes over all of the trials, given that each trial has a probability of success, P.

Here, a p-value for the ^{ith} k-mer (kmeri) is the probability that we would have seen a k-mer count |kmeri| at least this high (>=) in the FG, if all of the k-mers in the FG were sampled at random from the same underlying k-mer distribution as the BG.

For this experiment, a trial is choosing a k-mer at random from the background distribution of k-mers. The total number of trials (N) is represented by the total number of k-mers present in the foreground sample (the total number of times that a kmer was chosen at random from the background). In each trial, the probability of choosing a specific k-mer from the background (p) is given by its relative frequency in the background. The number of successes (X) is the number of times the specific k-mer was chosen from the background over all of the trials performed (again a trial here is picking a k-mer at random from the background sample). The probability of choosing a specific k-mer X # of times is what the binomial distribution calculates for each value of X.

p−valueᵢ=P(|kmerᵢ in FG|≤X≤Σᵢ|kmerᵢ in FG|)

Substrate samples can also be used as a BG to validate the statistical significance of k-mer enrichment between substrates.

To calculate the p-value for each k-mer in the FG, the binomial distribution was used to find the individual probabilities for all values of X between the count of the k-mer we saw in the foreground and the total number of k-mers in the FG (the maximum number of times we could have chosen the specific k-mer from the BG). The sum of all of those individual probabilities is the probability P(|specific kmer count in FG|<=X<=|total count of all kmers in FG|).

### Example 3. Protocol for Aptamer Fingerprinting

### Materials

Probe libraries.

| | |
|---|---|
| SB.PLA.LibProbe. | /5Phos/GAAACGGAGACGGATAAGACACAAAAAAACAAACAAA |
| 34L5P | AACCCC(N:25252525)(N)(N)(N)(N)(N)(N)(N)(N)(N)(N)(N)(N)(N)(N) |
| | GTAAAGGGAAAACAATGATACGAACACGCATA (SEQ ID NO: 1) |
| SB.PLAprimer 3F | GAAACGGAGACGGATAAGAC (SEQ ID NO: 2) |
| SB.PLAprimer 4R | TATGCGTGTTCGTATCATTG (SEQ ID NO: 3) |
| SB.PLA.LibProbe. | TATTGCGATAGCTGAGAGAGAAGACGCGAGGG(N:25252525)(N) |
| 12L3OH | (N)(N)(N)(N)(N)(N)(N)(N)(N)(N)(N)(N)(N)GCGAAAACAAAAAACA |
| | AAAATAAGAATCCAAGCAGCAGCAACA (SEQ ID NO: 4) |
| SB.PLAprimer 1F | TATTGCGATAGCTGAGAGAG (SEQ ID NO: 5) |
| SB.PLAprimer 2R | GTTGCTGCTGCTTGGATTC (SEQ ID NO: 6) |

N:25252525 refers to the equimolar ratios of the four nucleotides.

Folding, Annealing, Washing, and Binding buffer.

1X PBS, pH 7.4 with 5 mM MgCl2 (PBSM)

137 mM NaCl
2.7 mM KCl
10 mM Na₂HPO₄
1.8 mM KH₂PO₄

Prepare a 10× stock of the PBS buffer and, if necessary, adjust pH using hydrochloric acid or sodium hydroxide. Sterilize by filtration through 0.22 μm filter or by autoclaving at 15 psi for 20 min in the liquid cycle.
Eppendorf DNA lobind tubes 1.5 mL.
Oligo substrate 1

| | |
|---|---|
| SB.PLA.oligo. | AAGACCTACCTCACATGGCCAACACTCGGAC |
| subs.bio | AAAAAAAAAA/3Bio/ (SEQ ID NO: 7) |
| SB.bead.dT | TTTTTTTTTT/3InvdT/ (SEQ ID NO: 8) |

Streptavidin magnetic beads (NEB)

PCR reagents and 0.2 mL PCR tubes

### Method

Anneal SB.PLA.oligo.subs.bio and SB.bead.dT oligos (skip if using only biotin oligo):

| | | | |
|---|---|---|---|
| 100 uM SB.PLA.oligo.subs.bio | 10 | uL | Final conc = |
| | | | 20 uM |
| 100 uM SB. Bead.dT oligo | 10 | uL | Final conc = |
| | | | 20 uM |
| 10X PBS buffer | 10 | uL | Final conc = |
| | | | 1X |
| 100 mM MgCL2 | 5 | uL | Final conc = |
| | | | 5 mM |
| water | 65 | uL | |

1 min at 95 C. followed by slow cooling to 25 C. at 0.1 C./sec

Fold probe library (10×)

| | | |
|---|---|---|
| 100 uM probe library | 10 | Final concentration |
| | | 10 uM |
| 10X PBS | 10 | |
| 100 mM MgCL2 | 5 | |
| Water | 75 | |

1 min at 95 C. followed by slow cooling to 25 C. at 0.1 C./sec

Coat streptavidin beads with substrate oligo:

sVortex streptavidin beads and aliquot 1 uL into 1.5 mL eppendorf tubes
Wash beads 2X with 20 uL PBSM buffer.
Resuspend beads in 10 uL of annealed substrate oligo or PBSM buffer
Incubate at room temperature on rotator for 30 min
Wash beads 5X with 20 uL PBSM buffer

Bind probe libraries to substrates:

| | | |
|---|---|---|
| Make 50-fold dilution of the folded probe library: | | |
| Folded probe | 2 uL | Final concentration: |
| | | 0.2 uM |
| PBSM buffer | 98 uL | |
| Apply probes to the washed beads: | | |
| Washed beads + 10 uL (=2 pmol) folded probe | | |
| Incubate at room temperature on rotator for 30 min | | |

Wash beads:
For probe control samples use the entire 10 uL volume (beads+applied probes) for PCR in the next step
For zero wash sample, collect the beads on a magnet and replace the 10 uL supernatant with fresh 10 uL PBSM buffer
For the remaining beads perform 3× or 7× washes with 20 uL PBSM buffer and then resuspend the beads in total 10 uL PBSM buffer

Perform 100 uL PCR (maybe 50 uL PCR will work too):

| | volume | | |
|---|---|---|---|
| | (uL) | | |
| PCR reaction set up | | | |
| Beads | 10 | | |
| 10X thermopol buffer (NEB) | 10 | | |
| 4 mm dNTP | 5 | | |
| 20 uM fwd primer (~120 ng/ul) | 2 | | |
| 20 uM Rev primer | 2 | | |
| Taq DNA pol (5 U/ul stock: NEB | 1 | | |
| Water | 70 | | |
| PCR cycling conditions | | | |
| 1 | 95 | 2 | min |
| 2 | 95 | 10 | s |
| 3 | 55 | 15 | s |
| 4 | 72 | 30 | s |
| 20 cycles of steps 2 to 4 | | | |

Cycle number may need to be optimized by performing cycle course

### Example 4. iSeq PCR Library Protocol

Perform 100 uL PCR (maybe 50 uL PCR will work too):

| | Volume | | |
|---|---|---|---|
| | (uL) | *2.5 | |
| PCR reaction set up | | | |
| Beads (sample) | 10 | separate | |
| 10X thermopol buffer (NEB | 10 | 25 | |
| 4 mm dNTP | 5 | 12.5 | |
| 2 uM inner forward primer | 2 | 5 | |
| (~120 ng/ul) | | | |
| 2 uM inner Reverse primer | 2 | 5 | |
| 20 uM outer forward primer | 2 | 5 | |
| 20 uM outer forward primer | 2 | 5 | |
| Taq DNA pol (5 U/ul stock: NEB) | 1 | 2.5 | |
| Water | 66 | 165 | |
| | | 90 uL | |
| | | aliquot | |
| PCR cycling conditions | | | |
| 1 | 95 | 2 | min |
| 2 | 95 | 10 | s |
| 3 | 55 | 15 | s |
| 4 | 72 | 30 | s |

30 cycles of steps 2 to 4.
Gel purify 50 uL to 100 uL of the PCR using agarose gels.

Primer combinations used:

| | | |
|---|---|---|
| oligo probes | SB.NGSTSBC01F | SB.NGSTSBC05R |
| oligo 0 wash | SB.NGSTSBC01F | SB.NGSTSBC06R |
| oligo 3 wash | SB.NGSTSBC01F | SB.NGSTSBC07R |
| oligo 7 wash | SB.NGSTSBC01F | SB.NGSTSBC08R |
| SA probes | SB.NGSTSBC01F | SB.NGSTSBC05R |
| SA 0 wash | SB.NGSTSBC01F | SB.NGSTSBC06R |
| SA 3 wash | SB.NGSTSBC01F | SB.NGSTSBC07R |
| SA 7 wash | SB.NGSTSBC01F | SB.NGSTSBC08R |

iSeq pool.

| | | expected | ng in 1 | picomole/ | Concentration | conc |
|---|---|---|---|---|---|---|
| | ng/uL | bp | pmole | uL | (uM) | (nM) |
| Oligo probes | 36 | 222 | 147 | 0.25 | 0.25 | 246 |
| Oligo 0 wash | 38 | 222 | 147 | 0.26 | 0.26 | 259 |
| Oligo 3 wash | 34 | 222 | 147 | 0.23 | 0.23 | 232 |
| Oligo 7 was | 34 | 222 | 147 | 0.23 | 0.23 | 232 |
| Prep all dilutions in | | | | | | |
| DNA lobind tubes | | | | | | |
| | Dilute | 10 mM | | Dilute | 10 mM | |
| | to 40 nM | Tris | | to 4 nM | Tris | |
| | | pH 8.5 | | | pH 8.5 | |
| | 3.3 | 16.7 | | 10 | 90 | |
| | 3.1 | 16.9 | | 10 | 90 | |
| | 3.4 | 16.6 | | 10 | 90 | |
| | 3.4 | 16.6 | | 10 | 90 | |
| | Dilute | 10 mM | | | Dilute | |
| | to 400 pM | Tris | | | to 40 pM | |
| | | pH 8.5 | | | | |
| | 10 | 90 | | 400 pM | 10 | |
| | | | | Lib 1 | | |
| | 10 | 90 | | 400 pM | 10 | |
| | | | | Lib 2 | | |
| | 10 | 90 | | 400 pM | 10 | |
| | | | | Lib 3 | | |
| | 10 | 90 | | 400 pM | 10 | |
| | | | | Lib 4 | | |
| | | | | 400 pM | 10 | |
| | | | | PhiX | | |
| phiX v3 10 nM | Dilute | | | 10 mM | 50 | |
| | to 400 pM | | | Tris | | |
| | | | | pH 8.5 | | |
| | 4 | 96 uL | | | 0.2 | nM total |
| | | Tris | | | | concentration |
| Load 20 uL on iSeq | | | | | | |
| cartridge | | | | | | |

Primers

| | | | | | | |
|---|---|---|---|---|---|---|
| SB.PLA. | TCCCTACACGACGCTCT | Inner forward | | | | |
| NGS.1F | TCCGATCTTATTGCGAT | primer for Probe | | | | |
| | AGCTGAGAGAG (SEQ ID | 12 library | | | | |
| | NO: 9) | | | | | |
| SB.PLA. | GTTCAGACGTGTGCTCT | Inner reverse | | | | |
| NGS.2R | TCCGATCTGTTGCTGCT | primer for Probe | | | | |
| | GCTTGGATTC (SEQ ID | 12 library | | | | |
| | NO: 10) | | | | | |
| SB.PLA. | TCCCTACACGACGCTCT | Inner forward | | | | |
| NGS.3F | TCCGATCTGAAACGGA | primer for Probe | | | | |
| | GACGGATAAGAC (SEQ | 34 library | | | | |
| | ID NO: 11) | | | | | |
| SB.PLA. | GTTCAGACGTGTGCTCT | Inner reverse | | | | |
| NGS.4R | TCCGATCTTATGCGTGT | primer for Probe | | | | |
| | TCGTATCATTG (SEQ ID | 34 library | | | | |
| | NO: 12) | | | | | |
| SB.NGS | AATGATACGGCGACC | Universal outer | 68 bp | P5 | i5 | index rev |
| TSBC01F | ACCGAGATCTACACAT | Forward primer | | | | complement |
| | CACGACACTCTTTCCCT | | | | | |
| | ACACGACGCTCTTCCGA | | | | | |
| | TCT (SEQ ID NO: 13) | | | | | |
| SB.NGS | CAAGCAGAAGACGGC | Universal outer | 64 bp | P7 | i7 | CACTGT |
| TSBC05R | ATACGAGATACAGTGG | Reverse primer | | | | |
| | TGACTGGAGTTCAGAC | | | | | |
| | GTGTGCTCTTCCGATCT | | | | | |
| | (SEQ ID NO: 14) | | | | | |
| SB.NGS | CAAGCAGAAGACGGC | Universal outer | | | | ATTGGC |
| TSBC06R | ATACGAGATGCCAATG | Reverse primer | | | | |
| | TGACTGGAGTTCAGAC | | | | | |
| | GTGTGCTCTTCCGATCT | | | | | |
| | (SEQ ID NO: 15) | | | | | |
| SB.NGS | CAAGCAGAAGACGGC | Universal outer | | | | GATCTG |
| TSBC07R | ATACGAGATCAGATCG | Reverse primer | | | | |
| | TGACTGGAGTTCAGAC | | | | | |
| | GTGTGCTCTTCCGATCT | | | | | |
| | (SEQ ID NO: 16) | | | | | |
| SB.NGS | CAAGCAGAAGACGGC | Universal outer | | | | TCAAGT |
| TSBC08R | ATACGAGATACTTGAGT | Reverse primer | | | | |
| | GACTGGAGTTCAGACG | | | | | |
| | TGTGCTCTTCCGATCT | | | | | |
| | (SEQ ID NO: 17) | | | | | |

### Example 5. PLA Protocol

1 uM Phosphorylated library probe 34 in TE 10:0.1 (10 mM Tris, pH 7.5+0.1 mM EDTA)+50 mM NaCl (TENaB)

1 uM OH library probe 12 in TENaB

Probe refolding:
Using a thermocycler, incubate required volume of each probe at 95C for 1 min followed by slow cooling at 0.1 C/sec to 25C.

Surface prep.
Take 1 ul SA-Mag, 5 ul chitin-Mag, or 1 ul protein-G-Mag (NEB) beads after vortexing into 1.5 ml eppi tubes
Also take 3 mm discs of PES and PTR55GF papers
Wash 3× with 10 uL (beads) or 20 uL (paper) TENaB. Quick Vortex and 1.5 min on magnet each time for the beads. For paper do a quick spin.
Remove all wash buffer before adding next round of wash buffer or eventually probe mix
To each tube add 1+1+1 ul of 1 uM stock library probes 12 and 34P and TENaB Binding. Incubate on Rotator at room temp for 2 h to allow binding.

Washing.
For beads: wash 7× with 20 ul 1× TENaB each. Quick Vortex and 1.5 min on magnet each time and remove the buffer
For paper: wash 7× with 20 ul 1× TENaB. Quick Vortex, quick spin and remove buffer from paper

Ligation.

| | ul | *7 |
|---|---|---|
| Splint 2 (0.1 um) annealed 1:2 | 1 | 7 |
| with blocker 2 (in TENaB) | | |
| 10X T4 DNA ligase buffer (thawed | 3 | 21 |
| only once before for aliquoting) | | |
| T4 DNA Ligase (0.04 units/uL | 0.2 | 1.4 |
| dilution in 1X T4 ligase buffer) | | |
| PLA mix | beads or paper | separate |
| water | 25.8 | 180.6 |
| Incubate at room temp on rotator | total volume 30 ul; | |
| for required 10 min | ALIQUOT 30 UL INTO | |
| | washed PLA substrates | |

Then immediately incubate in Thermomixer set at 95 C. for 5 min
Then keep ligated products on ice till ready to add PCR mix

POR mix.

| | | |
|---|---|---|
| PLA ligation mix | 30 | |
| 10X thermopol buffer | 5 | 35 |
| 4 mM dNTP | 2.5 | 17.5 |
| 20 uM fwd primer primer1 | 0.5 | 3.5 |
| 20 uM Rev primer4 | 0.5 | 3.5 |
| Taq DNA pol 5 U/ul | 0.5 | 3.5 |
| Evagreen | 1.25 | 8.75 |
| water | 9.75 | 68.25 |

Aliquot 20 uL PCR mix to each of the 30 ul ligation mixes and transfer to PCR tubes (all on ice or cold block)

POR cycling.

| | | | | |
|---|---|---|---|---|
| 1 | 95 | 2 | min | |
| 2 | 95 | 15 | s | |
| 3 | 55 | 10 | s | 45 CYCLES |
| 4 | 72 | 30 | s | |

Max ramp times on the ABI Proflex PCR machine.

Gel purification.

Run entire PCR on 2% agarose gel
Gel purify desired band using Promega Wizard SV column. Elute in max 35 uL water
Nanodrop using 2 uL sample
NextGen Seq

Splint annealing.

| | |
|---|---|
| 1 um splint 2 | 5 |
| 1 um c2 | 10 |
| 5 m nacl | 0.5 |
| te 10:0.1 | 34.5 |

95 C. for 1 min then 0.1 c/sec to 25 C. (splint 2 alone not annealed)

Sequences.

| | |
|---|---|
| SB.PLA. | /5Phos/GAAACGGAGACGGATAAGACACAAAAAAACAAACAAAAACCC |
| LibProbe. | C(N:25252525)(N)(N)(N)(N)(N)(N)(N)(N)(N)(N)(N)(N)(N)(N)GTAAAGGGA |
| 34L5P | AAACAATGATACGAACACGCATA (SEQ ID NO: 1) |
| SB.PLA. | TATTGCGATAGCTGAGAGAGAAGACGCGAGGG(N:25252525)(N)(N)(N) |
| LibProbe. | (N)(N)(N)(N)(N)(N)(N)(N)(N)(N)(N)GCGAAAACAAAAAACAAAAATAAGA |
| 12L3OH | ATCCAAGCAGCAGCAACA (SEQ ID NO: 2) |
| splint | TCCGTTTCTGTTGCTG/3InvdT/ (SEQ ID NO: 18) |
| 2.3.16 | |
| SB.splint | CAACAGAAAC/3InvdT/ (SEQ ID NO: 19) |
| 2.c2 | |
| SB.PLA | TATTGCGATAGCTGAGAGAG (SEQ ID NO: 5) |
| primer 1F | |
| SB.PLA | GTTGCTGCTGCTTGGATTC (SEQ ID NO: 6) |
| primer 2R | |
| SB.PLA | GAAACGGAGACGGATAAGAC (SEQ ID NO: 2) |
| primer 3F | |
| SB.PLA | TATGCGTGTTCGTATCATTG (SEQ ID NO: 3) |
| primer 4R | |

1. A method of detecting a target in a sample, said method comprising:
obtaining a first library of probes and/or a second library of probes, wherein each probe of the first library comprises a 5′ primer binding site, a binding moiety, and a 3′ splinting site, wherein each probe of the second library comprises a 5′ splinting site, a binding moiety, and a 3′ primer binding site;
mixing the probes of the first library and/or the probes of the second library with the sample thereby allowing binding of the probes through their binding moieties to one or more substrates on the target;
ligating the 3′ splinting site of the probe of the first library and the splinting site of the probe of the second library thereby producing a ligated probe template;
performing amplification of the ligated probe template thereby producing a plurality of amplicons; and
sequencing the plurality of amplicons.

2. The method of claim 1, further comprises washing the sample before the step of ligation thereby removing the unbound probes.

3. The method of claim 1, wherein the binding moiety is a peptide, a nucleic acid, or an oligourethane.

4. The method of claim 1, wherein the binding moiety comprises a random sequence.

5. The method of claim 1, wherein the sequence of the primer binding site is unique to the probe.

6. The method of claim 1, wherein the probe further comprises a unique polynucleotide barcode.

7. The method of claim 1, wherein the binding moiety specifically binds to an organic or inorganic substrate.

8. The method of claim 1, wherein the splinting site is about 8 nucleotides in length.

9. The method of claim 1, wherein the splinting site is single stranded, a duplex, or a hemiduplex.

10. The method of claim 1, further comprising adding an oligonucleotide splint to the sample, wherein said oligonucleotide splint comprises a first region and a second region, wherein the first region is complementary to the 3′ splinting site of the probe of the first library and the second region is complementary to the 5′ splinting site of the probe of the second library.

11. The method of claim 10, wherein the ligation is with a T4 DNA ligase.

12. The method of claim 10, wherein the oligonucleotide splint is about 16 nucleotides in length.

13. The method of claim 1, wherein the amplicon is about at least 180 nucleotides in length.

14. The method of claim 1, further comprising identifying and quantifying the sequenced data.

15. The method of claim 14, wherein identifying and quantifying the sequenced data comprising extracting the sequences from the sequenced amplicons.

16. The method of claim 15, wherein the extracted sequences comprise the barcode sequences.

17. The method of claim 15, wherein the extracted sequences comprise the binding moiety sequences.

18. The method of claim 15, further comprising
determining a plurality of k-mers of each sequence;
determining the counts of each k-mer; and
calculating the p-value by comparing the frequency of each k-mer in the sample with a reference control.

19. The method of claim 18, wherein the p-value is calculated using binomial distribution.

20. The method of claim 14, further comprising translating the quantitative results into a graphic pattern.

21. The method of claim 20, wherein the graphic pattern represents the p-value of a k-mer and the sequence information of the k-mer.

22. The method of claim 20, further comprising comparing the graphical pattern to a reference control thereby identifying the target.

23. The method of claim 14, further comprising processing the qualified sequencing data using a dimensionality reduction algorithm.

24. The method of claim 23, wherein the dimensionality reduction algorithm is principal component analysis (PCA) or t-distributed stochastic neighbor embedding (t-SNE).

25-72. (canceled)
